(19) United States
(12) Patent Application Publication
(10) Pub. No.: US 2025/0236838 A1
(43) Pub. Date: Jul. 24, 2025

(54) E. COLI NISSLE STRAIN WITH IMPROVED TRANSFORMATION EFFICIENCY

(71) Applicant: Iowa State University Research Foundation, Inc., Ames, IA (US)

(72) Inventors: Thomas J. Mansell, Ames, IA (US); Chinmayi R. Gudi, Ames, IA (US)

(21) Appl. No.: 19/036,754

(22) Filed: Jan. 24, 2025

Related U.S. Application Data
(60) Provisional application No. 63/624,525, filed on Jan. 24, 2024.

(51) Int. Cl. C12N 1/20; A23L 33/135; C12N 9/22; C12N 15/113; C12N 15/87; C12R 1/19

(52) U.S. Cl. CPC C12N 1/205; A23L 33/135; C12N 9/22; C12N 15/113; C12N 15/87; C12N 2310/20; C12R 2001/19

(57) ABSTRACT

Disclosed are engineered E. coli Nissle strains having increased transformation efficiency. Also described are methods for generating transformants using the strains. Probiotic compositions containing the engineered E. coli Nissle strains and methods of using the probiotic compositions are also described herein.

### CROSS REFERENCE TO RELATED APPLICATIONS

This application claims priority to provisional application U.S. Ser. No. 63/624,525, filed Jan. 24, 2024, which is incorporated herein by reference in its entirety.

### GOVERNMENT SUPPORT

This invention was made with government support under R35 GM143074-01 awarded by the National Institutes of Health. The government has certain rights in the invention.

### SEQUENCE LISTING XML

The instant application contains a sequence listing, which has been submitted in XML file format by electronic submission and is hereby incorporated by reference in its entirety. Said XML file, created on Jan. 16, 2025, is named P14583US01.xml and is 10,865,159 bytes in size.

### TECHNICAL FIELD

The present disclosure relates generally to bacterial strains capable of improved transformation efficiencies.

### BACKGROUND

In recent years, the spotlight has increasingly focused on the gastrointestinal microflora, driving scientific, veterinary, and medical research interest. Consequently, probiotics, as live biotherapeutic agents, have garnered substantial attention. Among these, E. coli Nissle (EcN), a non-pathogenic gut isolate bacterium, is quickly gaining popularity. However, a formidable bottleneck in harnessing potential of EcN has been its poor transformation efficiency relative to other bacterial strains.

### SUMMARY

The need outlined above is met by the present disclosure which, in various embodiments, provides a novel engineered strain of E. coli Nissle, developed through adaptive laboratory evolution, showcasing a remarkable enhancement in transformation efficiency.

Engineered E. coli Nissle strains having increased transformation efficiency relative to wild type E. coli Nissle and derivatives thereof are provided. Probiotic compositions comprising the engineered E. coli Nissle strains of the disclosure are also provided.

Methods for establishing or maintaining a healthy gastrointestinal microbiota or reducing the effects of a gastrointestinal disorder in a subject comprising administering to the subject the engineered E. coli Nissle strains or probiotic compositions of the disclosure are provided.

Methods of obtaining an E. coli transformant comprising transforming a heterologous polynucleotide into the engineered E. coli Nissle strains of the disclosure are also provided.

Methods of altering a target nucleic acid sequence within an E. coli cell comprising providing a cell of an engineered E. coli Nissle strain of the disclosure with a Cas protein, a guide RNA, a donor nucleic acid sequence, and a recombinase are provided.

While multiple embodiments are disclosed, still other embodiments of the present disclosure will become apparent based on the detailed description, which shows and describes illustrative embodiments of the disclosure. Accordingly, the figures and detailed description are to be regarded as illustrative in nature and not restrictive.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The following drawings form part of the specification and are included to further demonstrate certain embodiments. In some instances, embodiments can be best understood by referring to the accompanying figures in combination with the detailed description presented herein. The description and accompanying figures may highlight a certain specific example, or a certain embodiment. However, one skilled in the art will understand that portions of the example or embodiment may be used in combination with other examples or embodiments.

FIG. 1A is a schematic representation of the protocol for the engineered strain of EcN.

FIG. 1B shows the transformation efficiency of the two strains with plasmids of various ORIs.

FIG. 2 shows superimposed images of the motility assay run for 16 hours for both strains. The Zapped strain is tagged Scarlet, and the wildtype is Neon Green.

FIG. 3A-C shows growth curves of wildtype and engineered (Zapped) EcN in various concentrations of ethanol (FIG. 3A), different values of pH (FIG. 3B), and different concentrations of sucrose (FIG. 3C).

FIG. 4 is a schematic representation of assay to find hydrophobicity of E. coli cells.

FIG. 5 shows average amount of plasmid DNA miniprepped from the Zapped and wildtype strains.

FIG. 6A-B shows the average amount of plasmid DNA made by the Zapped and wildtype strains.

FIG. 7 shows growth assays in iron enriched media.

FIG. 8 shows transformation efficiencies using heat-shock transformation.

FIG. 9A shows ratios of the impact of the mutations identified from whole genome sequencing. FIG. 9B shows the breakdown of INDELs and SNPs. FIG. 9C shows the percentages of variants that were predicted to have a significant impact.

### DETAILED DESCRIPTION

So that the present disclosure may be more readily understood, certain terms are first defined. Unless defined otherwise, all technical and scientific terms used herein have the same meaning as commonly understood by one of ordinary skill in the art to which embodiments of the disclosure pertain. Many methods and materials similar, modified, or equivalent to those described herein can be used in the practice of the embodiments of the present disclosure without undue experimentation, the preferred materials and methods are described herein. In describing and claiming the embodiments of the present disclosure, the following terminology will be used in accordance with the definitions set out below.

It is to be understood that all terminology used herein is for the purpose of describing particular embodiments only, and is not intended to be limiting in any manner or scope. For example, as used in this specification and the appended claims, the singular forms “a,” “an” and “the” can include plural referents unless the content clearly indicates otherwise. Similarly, the word “or” is intended to include “and” unless the context clearly indicate otherwise. The word “or” means any one member of a particular list and also includes any combination of members of that list. Further, all units, prefixes, and symbols may be denoted in its SI accepted form.

Numeric ranges recited within the specification are inclusive of the numbers defining the range and include each integer within the defined range. Throughout this disclosure, various embodiments of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾. This applies regardless of the breadth of the range.

As used herein, the term “administering” refers to the placement of a composition as disclosed herein into a subject by a method or route which results in at least partial delivery of the bacteria at a desired site. Compositions comprising the bacteria disclosed herein can be administered by any appropriate route which results in an effective treatment in the subject.

A “biologically pure culture” refers to a culture of bacteria containing no other bacterial species in quantities sufficient to interfere with the replication of the culture or be detected by normal bacteriological techniques. Stated another way, it is a culture wherein virtually all of the bacterial cells present are of the selected strain.

As used herein, the term “endogenous gene” refers to a native gene in its natural location in the genome of an organism.

As used herein, the term “engineered” in the context of a cell refers to a cell that has been genetically modified from its native state. For instance, an engineered E. coli cell may have nucleotide insertions, nucleotide deletions, nucleotide rearrangements, and nucleotide modifications introduced into their DNA. These genetic modifications may be present in the chromosome of the cell, or on a plasmid in the cell.

As used herein, the term “expression” refers to the transcription and stable accumulation of sense (mRNA) or anti-sense RNA derived from a nucleic acid, and/or to translation of an mRNA into a polypeptide.

As used herein, the term “gene” refers to a nucleic acid fragment that encodes a protein or fragment thereof, optionally including regulatory sequences preceding (5′ non-coding sequences) and following (3′ non-coding sequences) the coding sequence.

bacteria.

As used herein, a “heterologous polynucleotide”, “heterologous sequence”, or “heterologous nucleic acid” refers to a nucleic acid sequence that is not normally found in a given cell in nature. As used herein, a heterologous sequence encompasses a nucleic acid sequence that is exogenously introduced into a given cell.

As used herein, the term “isolated” refers to a material that is substantially or essentially free from components that normally accompany it in its native state. For example, an isolated bacterial strain may refer to a bacterial strain that has been purified or removed from naturally or non-naturally occurring components that are present in its naturally occurring environment.

“Microbiome” refers to the genetic content of the communities of microbes that live in and on the human body, both sustainably and transiently, including eukaryotes, archaea, bacteria, and viruses (including bacterial viruses (i.e., phage)), wherein “genetic content” includes genomic DNA, RNA such as ribosomal RNA, the epigenome, plasmids, and all other types of genetic information.

“Microbiota” refers to the community of microorganisms that inhabit (sustainably or transiently) in and/or on a subject, (e.g., a mammal such as a human), including, but not limited to, eukaryotes (e.g., protozoa), archaea, bacteria, and viruses (including bacterial viruses, i.e., a phage).

A “nucleic acid” or “nucleic acid sequence” may be any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable nucleic acid molecules are DNA, including genomic DNA or cDNA. Other suitable nucleic acid molecules are RNA, including mRNA.

The term “operatively linked” includes having an appropriate transcription start signal (e.g., promoter) in front of the polynucleotide sequence to be expressed, and having an appropriate translation start signal (e.g., a Shine Delgarno sequence and a start codon (ATG)) in front of the polypeptide coding sequence and maintaining the correct reading frame to permit expression of the polynucleotide sequence under the control of the expression control sequence, and, optionally, production of the desired polypeptide encoded by the polynucleotide sequence. In some examples, transcription of a gene encoding a recombinant polypeptide as described herein is under the control of a promoter sequence (or other transcriptional regulatory sequence) which controls the expression of the nucleic acid in a cell-type in which expression is intended. It will also be understood that the gene encoding a recombinant polypeptide as described herein can be under the control of transcriptional regulatory sequences which are the same or which are different from those sequences which control transcription of the naturally-occurring form of a protein.

The terms “overexpression” or “overexpress”, as used herein refers to the expression of a functional nucleic acid, polypeptide or protein encoded by DNA in a host cell, wherein the nucleic acid, polypeptide or protein is either not normally present in the host cell, or wherein the nucleic acid, polypeptide or protein is present in the host cell at a higher level than that normally expressed from the endogenous gene encoding the nucleic acid, polypeptide or protein.

A “pharmaceutical composition”, as used herein, refers to a composition comprising an active ingredient (e.g., an E. coli cell) with other components such as a pharmaceutically suitable carrier.

As used herein, the term “pharmaceutically acceptable” or “pharmacologically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. Moreover, for animal (e.g., human) administration, it will be understood that compositions should meet sterility, pyrogenicity, general safety and purity standards as required by the FDA Office of Biological Standards.

As used herein, the term “pharmaceutically acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, disintegrating agents, binders, sweetening agents, flavoring agents, perfuming agents, protease inhibitors, plasticizers, emulsifiers, stabilizing agents, viscosity increasing agents, film forming agents, solubilizing agents, surfactants, preservative and antioxidants can also be present in the formulation. The terms such as “excipient”, “carrier”, “pharmaceutically acceptable excipient” or the like are used interchangeably herein.

As used herein, the terms “pharmaceutically effective” or “therapeutically effective” shall mean an amount of a composition that is sufficient to show a meaningful benefit, i.e., treatment, prevention, amelioration, or a decrease in the frequency of the condition or symptom being treated.

A “plasmid” or “vector” includes a nucleic acid construct designed for delivery to a host cell or transfer between different host cell. The nucleic acid incorporated into the plasmid can be operatively linked to an expression control sequence when the expression control sequence controls and regulates the transcription and translation of that polynucleotide sequence.

As used herein “preventing” or “prevention” refers to any methodology where the disease state does not occur due to the actions of the methodology (such as, for example, administration of a composition as described herein). In certain embodiments, it is understood that prevention can also mean that the disease is not established to the extent that occurs in untreated controls. For example, there can be a 5, 10, 15, 20, 25, 30, 35, 40, 50, 60, 70, 80, 90, or 100% reduction in the establishment of disease frequency relative to untreated controls. Accordingly, prevention of a disease (e.g., a gastrointestinal disease or disorder), encompasses a reduction in the likelihood that a subject will develop the disease, relative to an untreated subject (e.g., a subject who does not receive a composition as described herein).

“Probiotic”, as used herein, refers to a live, non-pathogenic microorganism, e.g., a bacterium, which can confer health benefits to a host organism. Certain species, strains, and/or subtypes of non-pathogenic bacteria are currently recognized as probiotic.

A “promoter” as used herein, refers to a nucleotide sequence that is capable of controlling the expression of a coding sequence or gene. Promoters are generally located 5′ of the sequence that they regulate. Promoters may be derived in their entirety from a native gene, or be composed of different elements derived from promoters found in nature, and/or comprise synthetic nucleotide segments. Those skilled in the art will readily ascertain that different promoters may regulate expression of a coding sequence or gene in response to a particular stimulus, e.g., in a cell-specific or tissue-specific manner, in response to different environmental or physiological conditions, or in response to specific compounds. Prokaryotic promoters are typically classified into two classes: inducible and constitutive. “Constitutive promoter” refers to a promoter that is capable of facilitating continuous transcription of a coding sequence or gene under its control and/or to which it is operably linked. An “inducible promoter” refers to a promoter that initiates increased levels of transcription of the coding sequence or gene under its control in response to a stimulus or an exogenous environmental condition. A “directly inducible promoter” refers to a regulatory region, wherein the regulatory region is operably linked to a gene encoding a protein or polypeptide, where, in the presence of an inducer of said regulatory region, the protein or polypeptide is expressed. An “indirectly inducible promoter” refers to a regulatory system comprising two or more regulatory regions, for example, a first regulatory region that is operably linked to a first gene encoding a first protein, polypeptide, or factor, e.g., a transcriptional regulator, which is capable of regulating a second regulatory region that is operably linked to a second gene, the second regulatory region may be activated or repressed, thereby activating or repressing expression of the second gene. Both a directly inducible promoter and an indirectly inducible promoter are encompassed by “inducible promoter.” For example, and without limitation, chemical agents, temperature, and light may be used for induction of the promoters contemplated herein.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. The terms “protein” and “polypeptide” as used herein refer to both large polypeptides and small peptides. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

The term “subject” refers to any organism or animal subject that is an object of a method or material, including mammals, e.g., humans, laboratory animals (e.g., primates, rats, mice, rabbits), livestock (e.g., pigs, cows, horses, sheep, and goats), and household pets (e.g., dogs, cats, and rodents). As used herein, a subject can refer to a human or a non-human. Synonyms used herein include “patient” and “animal”.

“Treatment,” “treat,” or “treating” means a method of reducing the effects of a disease or condition. Treatment can also refer to a method of reducing the disease or condition itself rather than just the symptoms. The treatment can be any reduction from pre-treatment levels and can be but is not limited to the complete ablation of the disease, condition, or the symptoms of the disease or condition. Therefore, in the disclosed methods, “treatment” can refer to a 10%, 20%, 30%, 40%, 50%, 60%, 70%, 80%, 90%, or 100% reduction in the severity of an established disease or the disease progression. For example, a disclosed method for reducing the effects of a gastrointestinal disorder is considered to be a treatment if there is a 10% reduction in one or more symptoms of the disease in a subject with a gastrointestinal disorder when compared to pre-treatment levels in the same subject or control subjects. Thus, the reduction can be a 10, 20, 30, 40, 50, 60, 70, 80, 90, 100%, or any amount of reduction in between as compared to native or control levels. It is understood and herein contemplated that “treatment” does not necessarily refer to a cure of the disease or condition, but an improvement in the outlook of a disease or condition (e.g., a gastrointestinal disorder).

The gut microbiome comprises of billions of microbes featuring up to 1000 species. It plays a vital role in regulating metabolic functions of the body, training the immune system, and fighting pathogens. The microbes that inhabit the gut have profound effects on the digestive, cardiovascular, endocrine, neurological, and nervous systems. A vast majority of diseases and medical conditions can be linked to the GI tract. Hence, manipulating the gut microbiome by restoring or diversifying the ecosystem is crucial in healthcare and medicine. One method of manipulating the gut microbiome is using pharmabiotics, such as prebiotics, probiotics, and synbiotics.

E. coli Nissle 1917 (EcN) is a non-pathogenic bacteria discovered by Alfred Nissle in 1917 while studying Salmonella in humans. EcN was found to compete with pathogenic enterobacteria for iron and hence, inhibits their growth in the gut. Since discovery, EcN has become a popular probiotic. It is being engineered towards applications in non-invasive diagnosis and biotherapeutics. The viability of EcN as a probiotic relies on several limiting factors. Firstly, EcN colonizes poorly in the human GI tract. The high stability of the chromosome causes EcN to resist genetic modification, making bioengineering difficult. This manifests as poor transformation efficiency and recombineering in EcN. Adaptive lab evolution (ALE) presents a novel strategy for harnessing natural selection towards achieving specific goals in bioengineering. Using this strategy, a strain of EcN was engineered that shows improved transformation efficiency. The strain is a first-of-its-kind attempt to alleviate concerns regarding the transformation efficiency in EcN. Described herein, inter alia, are engineered E. coli Nissle strains having improved transformation efficiency.

The genetic changes that underlie the improved performance of the E. coli Nissle strain were identified. In certain embodiments, the engineered E. coli Nissle strain comprises one or more (2, 3, 4, 5, 6, 7, 8, 9, 10, etc.) of the mutations set forth in Table 5. In certain embodiments, the engineered E. coli Nissle strain comprises all of the mutations set forth in Table 5.

In certain embodiments, the mutation is at a position corresponding to position 257307, 261611, 287006, 313346, 372997, 547536, 577005, 619073, 905646, 948388, 1100140, 1106624, 1155295, 1155975, 1207958, 1214983, 1217272, 1330104, 1330105, 1431503, 1511516, 1559408, 1642042, 1693592, 1738308, 1775453, 1797994, 1802903, 1833436, 1908015, 1946344, 1954709, 1972000, 2029609, 2072645, 2150037, 2180269, 2188100, 2203779, 2222419, 2231010, 2275579, 2290073, 2338206, 2343227, 2370965, 2469948, 2472325, 2534241, 2673940, 2722786, 2723200, 2727344, 2740342, 2823249, 2825576, 2925375, 2984481, 3091331, 3109405, 3131290, 3190080, 3234031, 3290228, 3318100, 3381051, 3394188, 3408230, 3408232, 3408236, 3408254, 3408260, 3408264, 3408265, 3408271, 3408282, 3408298, 3408302, 3408310, 3408312, 3408319, 3408320, 3408324, 3408327, 3408332, 3408334, 3408339, 3408343, 3408346, 3408350, 3408353, 3408361, 3408363, 3408367, 3408369, 3408384, 3408401, 3408457, 3408468, 3408471, 3408480, 3410228, 3410238, 3410240, 3410243, 3410249, 3413110, 3451296, 3520054, 3554413, 3604748, 3640318, 3764568, 3844856, 3873401, 3888054, 4048903, 4175223, 4208844, 4208848, 4208851, 4265268, 4341919, 4383425, 4404105, 4463623, 4463628, 4463630, 4463634, 4463637, 4463638, 4463640, 4463644, 4463645, 4463650, 4463653, 4463655, 4463658, 4463660, 4463664, 4463678, 4463679, 4463692, 4463745, 4463757, 4609507, 4610114, 4649091, 4709868, 4729061, 4753558, 4756638, 4826691, 4826713, 4826728, 4826746, 4826749, 4826752, 4827732, 4827736, 4827746, 4827747, 4827750, 4827753, 4827763, 4827766, 4827770, 4827772, 4827774, 4827776, 4827778, 4889725, 4902377, 4976107, 5000528, 5010102, 5041738, 5068216, 5077148, 5080926, 5113156, 5125848, 5153839, 5153866, 5153890, 5163940, 5191207, 5249790, 5249835, 5249841, 5249844, 5249853, 5249856, 5249862, 5249877, 5249881, 5249886, 5249889, 5249892, 5249896, 5249910, 5249916, 5249931, 5249941, 5249943, 5249946, 5249949, 5249955, 5249958, 5249961, 5249970, 5249977, 5249981, 5249984, 5249988, 5249997, 5250003, 5250006, 5250009, 5250018, 5250021, 5250027, 5250034, 5250042, 5250402, 5250405, 5250417, 5250420, 5250424, 5250426, 5250432, 5250434, 5250435, 5250438, 5250441, 5250443, 5250468, 5250477, 5250478, 5250480, 5250483, 5250492, 5250504, 5250505, 5250507, 5250522, 5250524, 5250528, 5250532, 5250534, 5250540, 5250549, 5250552, 5250561, 5250564, 5250573, 5250576, 5304113, 5304115, 5304118, 5304124, 5304250, 5332501, 5345202, 5345207, 5345240, 5345241, 5345243, 5345245, 5345258, 5345263, 5345273, 5345280, 5345284, 5345285, 5345294, 5346922, 5374165, 5374166, 5374167, 5374172, 5374175, 5374184, 5374190, 5374195, 5374199, 5374202, 5374203, 5376958, 5428111, or 5430346 of the wild type E. coli Nissle strain set forth in SEQ ID NO: 1.

In certain embodiments, the mutation is a change from ‘TG’ to ‘T’ at position 257307, from ‘C’ to ‘CA’ at position 261611, from ‘CG’ to ‘C’ at position 287006, from ‘TG’ to ‘T’ at position 313346, from ‘AC’ to ‘A’ at position 372997, from ‘TC’ to ‘T’ at position 547536, from ‘CA’ to ‘C’ at position 577005, from ‘CT’ to ‘C’ at position 619073, from ‘TC’ to ‘T’ at position 905646, from ‘TC’ to ‘T’ at position 948388, from ‘TC’ to ‘T’ at position 1100140, from ‘TG’ to ‘T’ at position 1106624, from ‘CG’ to ‘C’ at position 1155295, from ‘TG’ to ‘T’ at position 1155975, from ‘TG’ to ‘T’ at position 1207958, from ‘AG’ to ‘A’ at position 1214983, from ‘AG’ to ‘A’ at position 1217272, from ‘A’ to ‘ATTC’ at position 1330104, from ‘A’ to ‘AGTAAACCAC’ (SEQ ID NO: 3) at position 1330105, from ‘G’ to ‘T’ at position 1431503, from ‘TA’ to ‘T’ at position 1511516, from ‘TC’ to ‘T’ at position 1559408, from ‘TG’ to ‘T’ at position 1642042, from ‘G’ to ‘GT’ at position 1693592, from ‘TC’ to ‘T’ at position 1738308, from ‘T’ to ‘TA’ at position 1775453, from ‘TC’ to ‘T’ at position 1797994, from ‘C’ to ‘CT’ at position 1802903, from ‘TC’ to ‘T’ at position 1833436, from ‘C’ to ‘CA’ at position 1908015, from ‘TC’ to ‘T’ at position 1946344, from ‘TC’ to ‘T’ at position 1954709, from ‘C’ to ‘CA’ at position 1972000, from ‘G’ to ‘GT’ at position 2029609, from ‘G’ to ‘GT’ at position 2072645, from ‘CG’ to ‘C’ at position 2150037, from ‘CT’ to ‘C’ at position 2180269, from ‘CT’ to ‘C’ at position 2188100, from ‘TC’ to ‘T’ at position 2203779, from ‘TG’ to ‘T’ at position 2222419, from ‘TC’ to ‘T’ at position 2231010, from ‘TG’ to ‘T’ at position 2275579, from ‘AT’ to ‘A’ at position 2290073, from ‘TC’ to ‘T’ at position 2338206, from ‘CA’ to ‘C’ at position 2343227, from ‘TC’ to ‘T’ at position 2370965, from ‘TC’ to ‘T’ at position 2469948, from ‘TC’ to ‘T’ at position 2472325, from ‘TC’ to ‘T’ at position 2534241, from ‘TC’ to ‘T’ at position 2673940, from ‘TC’ to ‘T’ at position 2722786, from ‘TC’ to ‘T’ at position 2723200, from ‘TC’ to ‘T’ at position 2727344, from ‘TC’ to ‘T’ at position 2740342, from ‘TG’ to ‘T’ at position 2823249, from ‘GA’ to ‘G’ at position 2825576, from ‘TC’ to ‘T’ at position 2925375, from ‘C’ to ‘CA’ at position 2984481, from ‘GC’ to ‘G’ at position 3091331, from ‘TC’ to ‘T’ at position 3109405, from ‘TC’ to ‘T’ at position 3131290, from ‘TC’ to ‘T’ at position 3190080, from ‘TG’ to ‘T’ at position 3234031, from ‘CG’ to ‘C’ at position 3290228, from ‘CA’ to ‘C’ at position 3318100, from ‘TC’ to ‘T’ at position 3381051, from ‘TC’ to ‘T’ at position 3394188, from ‘C’ to ‘G’ at position 3408230, from ‘G’ to ‘A’ at position 3408232, from ‘G’ to ‘A’ at position 3408236, from ‘C’ to ‘T’ at position 3408254, from ‘C’ to ‘G’ at position 3408260, from ‘A’ to ‘T’ at position 3408264, from ‘G’ to ‘C’ at position 3408265, from ‘G’ to ‘A’ at position 3408271, from ‘A’ to ‘C’ at position 3408282, from ‘CACT’ to ‘C’ at position 3408298, from ‘GGCA’ to ‘G’ at position 3408302, from ‘C’ to ‘A’ at position 3408310, from ‘ACCACC’ to ‘A’ at position 3408312, from ‘C’ to ‘G’ at position 3408319, from ‘A’ to ‘AAAAAT’ at position 3408320, from ‘C’ to ‘CTCT’ at position 3408324, from ‘C’ to ‘A’ at position 3408327, from ‘A’ to ‘AGCCT’ at position 3408332, from ‘C’ to ‘CTGCTACGGCCTGGTGTTT’ (SEQ ID NO: 4) at position 3408334, from ‘C’ to ‘CACGCCACTTTTCCATT’ (SEQ ID NO: 5) at position 3408339, from ‘A’ to ‘ATATCT’ at position 3408343, from ‘AC’ to ‘A’ at position 3408346, from ‘C’ to ‘T’ at position 3408350, from ‘GTTACA’ to ‘G’ at position 3408353, from ‘G’ to ‘A’ at position 3408361, from ‘A’ to ‘AT’ at position 3408363, from ‘A’ to ‘C’ at position 3408367, from ‘G’ to ‘GT’ at position 3408369, from ‘T’ to ‘C’ at position 3408384, from ‘A’ to ‘C’ at position 3408401, from ‘T’ to ‘C’ at position 3408457, from ‘T’ to ‘C’ at position 3408468, from ‘C’ to ‘T’ at position 3408471, from ‘A’ to ‘G’ at position 3408480, from ‘G’ to ‘A’ at position 3410228, from ‘A’ to ‘G’ at position 3410238, from ‘T’ to ‘C’ at position 3410240, from ‘G’ to ‘A’ at position 3410243, from ‘C’ to ‘G’ at position 3410249, from ‘TG’ to ‘T’ at position 3413110, from ‘G’ to ‘GT’ at position 3451296, from ‘TC’ to ‘T’ at position 3520054, from ‘CA’ to ‘C’ at position 3554413, from ‘AG’ to ‘A’ at position 3604748, from ‘TC’ to ‘T’ at position 3640318, from ‘TC’ to ‘T’ at position 3764568, from ‘TC’ to ‘T’ at position 3844856, from ‘CG’ to ‘C’ at position 3873401, from ‘G’ to ‘T’ at position 3888054, from ‘TC’ to ‘T’ at position 4048903, from ‘AG’ to ‘A’ at position 4175223, from ‘AG’ to ‘A’ at position 4208844, from ‘A’ to ‘G’ at position 4208848, from ‘T’ to ‘C’ at position 4208851, from ‘CG’ to ‘C’ at position 4265268, from ‘TC’ to ‘T’ at position 4341919, from ‘G’ to ‘GT’ at position 4383425, from ‘TA’ to ‘T’ at position 4404105, from ‘T’ to ‘TGAGC’ at position 4463623, from ‘A’ to ‘AC’ at position 4463628, from ‘G’ to ‘GCAT’ at position 4463630, from ‘A’ to ‘C’ at position 4463634, from ‘T’ to ‘G’ at position 4463637, from ‘T’ to ‘TCGG’ at position 4463638, from ‘CA’ to ‘C’ at position 4463640, from ‘G’ to ‘C’ at position 4463644, from ‘C’ to ‘T’ at position 4463645, from ‘A’ to ‘AAAGTACCTGCATT’ (SEQ ID NO: 6) at position 4463650, from ‘G’ to ‘GAGA’ at position 4463653, from ‘T’ to ‘TCA’ at position 4463655, from ‘T’ to ‘TCGGCCAGG’ at position 4463658, from ‘T’ to ‘C’ at position 4463660, from ‘GAAAA’ to ‘G’ at position 4463664, from ‘A’ to ‘G’ at position 4463678, from ‘A’ to ‘AGC’ at position 4463679, from ‘T’ to ‘TCCACCATGTTTGAGTTTGTTCCGGAAAGTCTTTCCGGGTC’ (SEQ ID NO: 7) at position 4463692, from ‘A’ to ‘G’ at position 4463745, from ‘G’ to ‘A’ at position 4463757, from ‘A’ to ‘ACCGTGTACGTACAAGCAGTGGGAGCCTCTTAATGGGGTGACTGCGTACCTTTTGT ATAATGGGTCAGCGACTTATATTCTGTAGCAAGGTTAACCGAATAGGGGAGCCGAA GGGAAACCGAGTCTTAACTGGGCGTTAAGTTGCAGGGTATAGACCCGAAACCCGGT GATCTAGCCATGGGCAGGTTGAAGGTTGGGTAACACTAACTGGAGGACCGAACCGA CTAATGTTGAAAAATTAGCGGATGACTTGTGGCTGGGGGTGAAAGGCCAATCAAAC CGGGAGATAGCTGGTTCTCCCCGAAAGCTATTTAGGTAGCGCCTCGTGAATTCATCT CCGGGGGTAGAGCACTGTTTCGGCAAGGGGGTCATCCCGACTTACCAACCCGATGC AAACTGCGAATACCGGAGAATGTTATCACGGGAGACACACGGCGGGTGCTAACGTC CGTCGTGAAGAGGGAAACAACCCAGACCGCCAGCTAAGGTCCCAAAGTCATGGTTA AGTGGGAAACGATGTGGGAAGGCCCAGACAGCCAGGATGTTGGCTTAGAAGCAGC CATCATTTAAAGAAAGCGTAATAGCTCACTGGTCGAGTCGGCCTGCGCGGAAGATG TAACGGGGCTAAACCATGCACCGAAGCTGCGGCAGCGACACTATGTGTTGTTGGGT AGGGGAGCGTTCTGTAAGCCTGTGAAGGTGGCCTGTGAGGGTTGCTGGAGGTATCA GAAGTGCGAATGCTGACATAAGTAACGATAAAGCGGGTGAAAAGCCCGCTCGCCG GAAGACCAAGGGTTCCTGTCCAACGTTAATCGGGGCAGGGTGAGTCGACCCCTAAG GCGAGGCCGAAAGGCGTAGTCGATGGGAAACAGGTTAATATTCCTGTACTTGGTGT TACTGCGAAGGGGGGACGGAGAAGGCTATGTTGGCCGGGCGACGGTTGTCCCGGTT TAAGCGTGTAGGCTGGTTTTCCAGGCAAATCCGGAAAACCAAGGCTGAGGCGTGAT GACGAGGCACTACGGTGCTGAAGCGACAAATGCCCTGCTTCCAGGAAAAGCCTCTA AGCATCAGGTAACATCAAATCGTACCCCAAACCGACACAGGTGGTCAGGTAGAGAA TACCAAGGCGCTTGAGAGAACTCGGGTGAAGGAACTAGGCAAAATGGTGCCGTAA CTTCGGGAGAAGGCACGCTGATATGTAGGTGAAGCGACTTGCTCGTGGAGCTGAAA TCAGTCGAAGATACCAGCTGGCTGCAACTGTTTATTAAAAACACAGCACTGTGCAA ACACGAAAGTGGACGTATACGGTGTGACGCCTGCCCGGTGCCGGAAGGTTAATTGA TGGGGTTAGCGGTAACGCGAAGCTCTTGATCGAAGCCCCGGTAAACGGCGGCCGTA ACTATAACGGTCCTAAGGTAGCGAAATTCCTTGTCGGGTAAGTTCCGACCTGCACG AATGGCGTAATGATGGCCAGGCTGTCTCCACCCGAGACTCAGTGAAATTGAACTCG CTGTGAAGATGCAGTGTACCCGCGGCAAGACGGAAAGACCCCGTGAACCTTTACTA TAGCTTGACACTGAACATTGAGCCTTGATGTGTAGGATAGGTGGGAGGCTTTGAAG TGTGGACGCCAGTCTGCATGGAGCCGACCTTGAAATACCACCCTTTAATGTTTGATG TTCTAACGTTGGCCCGTAATCCGGGTTGCGGACAGTGTCTGGTGGGTAGTTTGACTG GGGCGGTCTCCTCCTAAAGAGTAACGGAGGAGCACGAAGGTTGGCTAATCCTGGTC GGACATCAGGAGGTTAGTGCAATGGCATAAGCCAGCTTGACTGCGAGCGTGACGGC GCGAGCAGGTGCGAAAGCAGGTCATAGTGATCCGGTGGTTCTGAATGGAAGGGCCA TCGCTCAACGGATAAAAGGTACTCCGGGGATAACAGGCTGATACCGCCCAAGAGTT CATATCGACGGCGGTGTTTGGCACCTCGATGTCGGCTCATCACATCCTGGGGCTGAA GTAGGTCCCAAGGGTATGGCTGTTCGCCATTTAAAGTGGTACGCGAGCTGGGTTTA GAACGT’ (SEQ ID NO: 8) at position 4609507, from ‘TC’ to ‘T’ at position 4610114, from ‘TC’ to ‘T’ at position 4649091, from ‘C’ to ‘CA’ at position 4709868, from ‘TC’ to ‘T’ at position 4729061, from ‘TG’ to ‘T’ at position 4753558, from ‘C’ to ‘T’ at position 4756638, from ‘G’ to ‘A’ at position 4826691, from ‘A’ to ‘T’ at position 4826713, from ‘G’ to ‘T’ at position 4826728, from ‘C’ to ‘T’ at position 4826746, from ‘T’ to ‘C’ at position 4826749, from ‘T’ to ‘C’ at position 4826752, from ‘AGT’ to ‘A’ at position 4827732, from ‘T’ to ‘TCG’ at position 4827736, from ‘G’ to ‘T’ at position 4827746, from ‘A’ to ‘C’ at position 4827747, from ‘T’ to ‘C’ at position 4827750, from ‘C’ to ‘T’ at position 4827753, from ‘G’ to ‘A’ at position 4827763, from ‘G’ to ‘A’ at position 4827766, from ‘C’ to ‘T’ at position 4827770, from ‘G’ to ‘A’ at position 4827772, from ‘A’ to ‘T’ at position 4827774, from ‘A’ to ‘T’ at position 4827776, from ‘G’ to ‘GGCGC’ at position 4827778, from ‘TG’ to ‘T’ at position 4889725, from ‘TC’ to ‘T’ at position 4902377, from ‘TC’ to ‘T’ at position 4976107, from ‘G’ to ‘GT’ at position 5000528, from ‘TC’ to ‘T’ at position 5010102, from ‘CA’ to ‘C’ at position 5041738, from ‘TC’ to ‘T’ at position 5068216, from ‘TC’ to ‘T’ at position 5077148, from ‘T’ to ‘C’ at position 5080926, from ‘CA’ to ‘C’ at position 5113156, from ‘T’ to ‘C’ at position 5125848, from ‘A’ to ‘G’ at position 5153839, from ‘G’ to ‘A’ at position 5153866, from ‘A’ to ‘T’ at position 5153890, from ‘CG’ to ‘C’ at position 5163940, from ‘G’ to ‘T’ at position 5191207, from ‘T’ to ‘C’ at position 5249790, from ‘T’ to ‘C’ at position 5249835, from ‘G’ to ‘T’ at position 5249841, from ‘G’ to ‘A’ at position 5249844, from ‘A’ to ‘G’ at position 5249853, from ‘C’ to ‘G’ at position 5249856, from ‘C’ to ‘G’ at position 5249862, from ‘A’ to ‘G’ at position 5249877, from ‘C’ to ‘T’ at position 5249881, from ‘T’ to ‘A’ at position 5249886, from ‘C’ to ‘G’ at position 5249889, from ‘T’ to ‘C’ at position 5249892, from ‘G’ to ‘A’ at position 5249896, from ‘C’ to ‘G’ at position 5249910, from ‘C’ to ‘T’ at position 5249916, from ‘C’ to ‘G’ at position 5249931, from ‘T’ to ‘C’ at position 5249941, from ‘A’ to ‘C’ at position 5249943, from ‘C’ to ‘A’ at position 5249946, from ‘C’ to ‘T’ at position 5249949, from ‘G’ to ‘A’ at position 5249955, from ‘T’ to ‘C’ at position 5249958, from ‘C’ to ‘G’ at position 5249961, from ‘C’ to ‘T’ at position 5249970, from ‘C’ to ‘T’ at position 5249977, from ‘C’ to ‘CG’ at position 5249981, from ‘TA’ to ‘T’ at position 5249984, from ‘G’ to ‘C’ at position 5249988, from ‘G’ to ‘A’ at position 5249997, from ‘T’ to ‘C’ at position 5250003, from ‘G’ to ‘C’ at position 5250006, from ‘G’ to ‘C’ at position 5250009, from ‘A’ to ‘G’ at position 5250018, from ‘T’ to ‘G’ at position 5250021, from ‘T’ to ‘C’ at position 5250027, from ‘C’ to ‘T’ at position 5250034, from ‘G’ to ‘A’ at position 5250042, from ‘C’ to ‘T’ at position 5250402, from ‘C’ to ‘T’ at position 5250405, from ‘T’ to ‘C’ at position 5250417, from ‘C’ to ‘G’ at position 5250420, from ‘C’ to ‘T’ at position 5250424, from ‘A’ to ‘G’ at position 5250426, from ‘T’ to ‘A’ at position 5250432, from ‘G’ to ‘A’ at position 5250434, from ‘C’ to ‘G’ at position 5250435, from ‘A’ to ‘G’ at position 5250438, from ‘T’ to ‘C’ at position 5250441, from ‘A’ to ‘G’ at position 5250443, from ‘T’ to ‘C’ at position 5250468, from ‘A’ to ‘G’ at position 5250477, from ‘A’ to ‘C’ at position 5250478, from ‘A’ to ‘G’ at position 5250480, from ‘G’ to ‘C’ at position 5250483, from ‘C’ to ‘T’ at position 5250492, from ‘G’ to ‘T’ at position 5250504, from ‘C’ to ‘A’ at position 5250505, from ‘G’ to ‘C’ at position 5250507, from ‘T’ to ‘TA’ at position 5250522, from ‘GC’ to ‘G’ at position 5250524, from ‘A’ to ‘G’ at position 5250528, from ‘A’ to ‘G’ at position 5250532, from ‘T’ to ‘C’ at position 5250534, from ‘C’ to ‘G’ at position 5250540, from ‘C’ to ‘T’ at position 5250549, from ‘T’ to ‘C’ at position 5250552, from ‘A’ to ‘G’ at position 5250561, from ‘G’ to ‘A’ at position 5250564, from ‘A’ to ‘G’ at position 5250573, from ‘T’ to ‘G’ at position 5250576, from ‘G’ to ‘A’ at position 5304113, from ‘A’ to ‘G’ at position 5304115, from ‘G’ to ‘A’ at position 5304118, from ‘T’ to ‘C’ at position 5304124, from ‘A’ to ‘C’ at position 5304250, from ‘CA’ to ‘C’ at position 5332501, from ‘T’ to ‘C’ at position 5345202, from ‘A’ to ‘G’ at position 5345207, from ‘C’ to ‘G’ at position 5345240, from ‘TC’ to ‘T’ at position 5345241, from ‘A’ to ‘T’ at position 5345243, from ‘G’ to ‘GA’ at position 5345245, from ‘A’ to ‘AC’ at position 5345258, from ‘CT’ to ‘C’ at position 5345263, from ‘A’ to ‘G’ at position 5345273, from ‘CCG’ to ‘C’ at position 5345280, from ‘G’ to ‘C’ at position 5345284, from ‘G’ to ‘GAC’ at position 5345285, from ‘A’ to ‘G’ at position 5345294, from ‘TG’ to ‘T’ at position 5346922, from ‘G’ to ‘T’ at position 5374165, from ‘G’ to ‘A’ at position 5374166, from ‘C’ to ‘A’ at position 5374167, from ‘A’ to ‘AG’ at position 5374172, from ‘TC’ to ‘T’ at position 5374175, from ‘G’ to ‘A’ at position 5374184, from ‘A’ to ‘C’ at position 5374190, from ‘C’ to ‘G’ at position 5374195, from ‘A’ to ‘T’ at position 5374199, from ‘G’ to ‘A’ at position 5374202, from ‘T’ to ‘A’ at position 5374203, from ‘TG’ to ‘T’ at position 5376958, from ‘AC’ to ‘A’ at position 5428111, from ‘G’ to ‘GTGCGTTTGCAGCAC’ (SEQ ID NO: 9) at position 5430346 of the wild type E. coli Nissle strain set forth in SEQ ID NO: 1.

In certain embodiments, the mutation is located in one or more genes selected from ECOLIN_01245, ECOLIN_01235, ECOLIN_01465, ECOLIN_01570, ECOLIN_01800, ECOLIN_02590, ECOLIN_02735, ECOLIN_02915, ECOLIN_04325, ECOLIN_04550, ECOLIN_05190, ECOLIN_05225, ECOLIN_05515, ECOLIN_05765, ECOLIN_05835, ECOLIN_05860, ECOLIN_06500, ECOLIN_07110, ECOLIN_07475, ECOLIN_07715, ECOLIN_08080, ECOLIN_08295, ECOLIN_08490, ECOLIN_08670, ECOLIN_08785, ECOLIN_08815, ECOLIN_09000, ECOLIN_09345, ECOLIN_09555, ECOLIN_09600, ECOLIN_09660, ECOLIN_09995, ECOLIN_10230, ECOLIN_10720, ECOLIN_10880, ECOLIN_10920, ECOLIN_10995, ECOLIN_11035, ECOLIN_11065, ECOLIN_11170, ECOLIN_11200, ECOLIN_11435, ECOLIN_11470, ECOLIN_11585, ECOLIN_12000, ECOLIN_12005, ECOLIN_12290, ECOLIN_12890, ECOLIN_13120, ECOLIN_13135, ECOLIN_13170, ECOLIN_13585, ECOLIN_13590, ECOLIN_13975, ECOLIN_14250, ECOLIN_14825, ECOLIN_15025, ECOLIN_15290, ECOLIN_15460, ECOLIN_15675, ECOLIN_15840, ECOLIN_16160, ECOLIN_16210, ECOLIN_16295, ECOLIN_16300, ECOLIN_16315, ECOLIN_16345, ECOLIN_16530, ECOLIN_16875, ECOLIN_17025, ECOLIN_17265, ECOLIN_17435, ECOLIN_18045, ECOLIN_18570, ECOLIN_18720, ECOLIN_18785, ECOLIN_19485, ECOLIN_20030, ECOLIN_20180, ECOLIN_20530, ECOLIN_20865, ECOLIN_21080, ECOLIN_21155, ECOLIN_21485, ECOLIN_22180, ECOLIN_22185, ECOLIN_22350, ECOLIN_22645, ECOLIN_22710, ECOLIN_22830, ECOLIN_22825, ECOLIN_23160, ECOLIN_23165, ECOLIN_23560, ECOLIN_23655, ECOLIN_23965, ECOLIN_24070, ECOLIN_24140, ECOLIN_24275, ECOLIN_24410, ECOLIN_24460, ECOLIN_24475, ECOLIN_24655, ECOLIN_24715, ECOLIN_24845, ECOLIN_24895, ECOLIN_25060, ECOLIN_25390, ECOLIN_25740, ECOLIN_25870, ECOLIN_25925, ECOLIN_25930, ECOLIN_26080, ECOLIN_26075, ECOLIN_26325, and ECOLIN_26350.

In certain embodiments, the engineered E. coli Nissle strain of the disclosure comprises a genome sequence having at least 80%, at least 85%, at least 90%, at least 95%, at least 98%, at least 99%, at least 99.5% at least 99.6% at least 99.7% at least 99.8%, at least 99.9%, or 100% sequence identity to SEQ ID NO: 2.

A sample of the engineered E. coli Nissle strain of the disclosure has been deposited with the American Type Culture Collection (ATCC), 10801 University Boulevard, Manassas, VA 20110-2209, U.S.A., under the Budapest Treaty on ______ and has been assigned the following accession number: PTA-______.

The strain has been deposited under conditions that assure that access to the strain will be available during the pendency of this patent application to one determined by the Commissioner of Patents and Trademarks to be entitled thereto under 37 CFR 1.14 and 35 USC 122. Further, the culture deposit will be stored and made available to the public in accord with the provisions of the Budapest Treaty for the Deposit of Microorganisms, i.e., it will be stored with all the care necessary to keep it viable and uncontaminated for a period of at least five years after the most recent request for the furnishing of a sample of the deposit, and in any case, for a period of at least 30 (thirty) years after the date of deposit or for the enforceable life of any patent which may issue disclosing the strain. The depositor acknowledges the duty to replace the deposit should the depository be unable to furnish a sample when requested, due to the condition of the deposit. All restrictions on the availability to the public of the culture deposit will be irrevocably removed upon the granting of a patent disclosing it.

Derivatives of the engineered E. coli Nissle strain are also provided. As used herein, the phrase “derivatives thereof”, when used in the context of an engineered E. coli Nissle strain, refers to any strain that is obtained from the engineered E. coli Nissle strain. Derivatives of an engineered E. coli Nissle strain include, but are not limited to, variants of the strain obtained by selection, variants of the strain selected by mutagenesis and selection, and a genetically transformed strain obtained from the engineered E. coli Nissle strain.

The engineered E. coli Nissle strains described herein are useful for producing E. coli transformants. In certain embodiments, a method of obtaining an E. coli transformant, comprising transforming a heterologous polynucleotide into an engineered E. coli Nissle strain of the disclosure is provided. In certain embodiments, a method of obtaining an E. coli transformant, comprising: (a) cultivating an engineered E. coli Nissle of the disclosure; (b) transforming a heterologous polynucleotide into the strain of (a); and (c) isolating the transformant strain resulting from (b) is provided.

The transformed DNA described herein can be any DNA of interest. The DNA may be of genomic, cDNA, semisynthetic, synthetic origin, or any combinations thereof. The DNA may be a heterologous polynucleotide that encodes any polypeptide having biological activity of interest or may be a DNA involved in the expression of the polypeptide having biological activity, e.g., a promoter.

The polypeptide having a biological activity may be any polypeptide of interest. The polypeptide may be native or foreign to the E. coli cell. The polypeptide may be naturally occurring allelic and engineered variations of the below-mentioned polypeptides and hybrid polypeptides.

The term “polypeptide” is not meant herein to refer to a specific length of the encoded product and, therefore, encompasses peptides, oligopeptides, and proteins. The term “polypeptide” also encompasses hybrid polypeptides, which comprise a combination of partial or complete polypeptide sequences obtained from at least two different polypeptides wherein one or more may be foreign to the E. coli cell. Polypeptides further include naturally occurring allelic and engineered variations of a polypeptide.

In certain embodiments, the polypeptide is an antibody, antigen, antimicrobial peptide, enzyme, growth factor, hormone, immunodilator, neurotransmitter, receptor, reporter protein, structural protein, or transcription factor.

In certain embodiments, the polypeptide is a hybrid polypeptide, which comprises a combination of partial or complete polypeptide sequences obtained from at least two different polypeptides wherein one or more may be foreign to the E. coli cell.

In certain embodiments, the polypeptide is a fused polypeptide in which another polypeptide is fused at the N-terminus or the C-terminus of the polypeptide or fragment thereof. A fused polypeptide is produced by fusing a nucleotide sequence (or a portion thereof) encoding one polypeptide to a nucleotide sequence (or a portion thereof) encoding another polypeptide. Techniques for producing fusion polypeptides are known in the art, and include, ligating the coding sequences encoding the polypeptides so that they are in frame and expression of the fused polypeptide is under control of the same promoter(s) and terminator.

The heterologous polynucleotide may be obtained from any prokaryotic, eukaryotic, or other source. Techniques used to isolate or clone a heterologous polynucleotide are known in the art and include isolation from genomic DNA, preparation from cDNA, or a combination thereof. The cloning of the DNA of interest from such genomic DNA can be effected, e.g., by using the well known polymerase chain reaction (PCR). See, for example, Innis et al., PCR Protocols: A Guide to Methods and Application, Academic Press, New York, 1990. The cloning procedures may involve excision and isolation of a desired nucleic acid fragment, insertion of the fragment into a vector molecule, and incorporation of the recombinant vector into the engineered E. coli strain where multiple copies or clones of the nucleic acid sequence will be replicated. The DNA may be of genomic, cDNA, RNA, semisynthetic, synthetic origin, or any combinations thereof.

A heterologous polynucleotide may be manipulated in a variety of ways to provide for expression in an engineered E. coli strain. Manipulation of the polynucleotide sequence prior to its insertion into a vector may be desirable or necessary depending on the expression vector. The techniques for modifying polynucleotide sequences utilizing recombinant DNA methods are well known in the art.

A nucleic acid construct comprising a polynucleotide encoding a polypeptide may be operably linked to one or more (e.g., two, several) control sequences capable of directing expression of the coding sequence in an engineered E. coli strain of the present disclosure under conditions compatible with the control sequences.

The control sequence may be an appropriate promoter sequence, a nucleotide sequence that is recognized by an engineered E. coli strain of the present disclosure for expression of the polynucleotide encoding the polypeptide. The promoter sequence contains transcriptional control sequences that mediate expression of the polypeptide. The promoter may be any nucleotide sequence that shows transcriptional activity in the engineered E. coli strain, including mutant, truncated, and hybrid promoters, and may be obtained from genes encoding extracellular or intracellular polypeptides either native or foreign to the engineered E. coli strain.

Examples of suitable promoters for directing the transcription of the nucleic acid constructs in an engineered E. coli strain include an E. coli σ^{s} promoter, a an E. coli σ³² promoter, an E. coli σ⁷⁰ promoter, a Bacillus subtilis σ^{A} promoter, a Bacillus subtilis σ^{B} promoter, a bacteriophage T7 promoter, an E. coli lac operon promotor, and E. coli trc promoter. Further promoters are described in “Useful proteins from recombinant bacteria” in Gilbert et al., Scientific American 1980, 242, 74-94; and in Sambrook et al., 1989, supra.

The control sequence may also be a suitable transcription terminator sequence, a sequence recognized by an engineered E. coli strain to terminate transcription. The terminator sequence is operably linked to the 3′ terminus of the nucleotide sequence encoding the heterologous polypeptide. Any terminator that is functional in a E. coli strain may be used.

The control sequence may also be a suitable leader sequence, a nontranslated region of mRNA that is important for translation by an engineered E. coli strain. The leader sequence is operably linked to the 5′ terminus of the nucleotide sequence encoding the heterologous polypeptide. Any leader sequence that is functional in the engineered E. coli strain may be used.

The control sequence may also be a signal peptide coding sequence that codes for an amino acid sequence linked to the amino terminus of a polypeptide and directs the encoded polypeptide into the cell's secretory pathway. The 5′ end of the coding sequence of the nucleotide sequence may inherently contain a signal peptide coding sequence naturally linked in translation reading frame with the segment of the coding sequence that encodes the secreted polypeptide. Alternatively, the 5′ end of the coding sequence may contain a signal peptide coding sequence that is foreign to the coding sequence. The foreign signal peptide coding sequence may be required where the coding sequence does not naturally contain a signal peptide coding sequence. Alternatively, the foreign signal peptide coding sequence may simply replace the natural signal peptide coding sequence in order to enhance secretion of the polypeptide. However, any signal peptide coding sequence that directs the expressed polypeptide into the secretory pathway of the engineered E. coli strain, i.e., secreted into a culture medium, may be used in the present disclosure.

A recombinant expression vector comprising a nucleotide sequence, a promoter, and transcriptional and translational stop signals may be used for the recombinant production of a polypeptide of interest. The various nucleic acids and control sequences described herein may be joined together to produce a recombinant expression vector that may include one or more (e.g., two, several) convenient restriction sites to allow for insertion or substitution of the nucleotide sequence encoding the polypeptide at such sites. Alternatively, the nucleotide sequence may be expressed by inserting the nucleotide sequence or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the nucleotide sequence. The choice of the vector will typically depend on its compatibility with the engineered E. coli strain into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the engineered E. coli strain, is integrated into the genome and replicated together with the chromosome(s) into which it has been integrated. Furthermore, a single vector or plasmid or two or more vectors or plasmids that together contain the total DNA to be introduced into the genome of the engineered E. coli strain, or a transposon, may be used.

The vector may contain one or more (e.g., two, several) selectable markers that permit easy selection of transformed engineered E. coli strains. A selectable marker is a gene the product of which provides for biocide or viral resistance, resistance to heavy metals, prototrophy to auxotrophs, and the like.

Examples of selectable markers for use in the engineered E. coli strain include, but are not limited to, markers that confer antibiotic resistance such as ampicillin, chloramphenicol, kanamycin, or tetracycline resistance.

The vectors may contain one or more (e.g., two, several) elements that permit integration of the vector into the E. coli genome or autonomous replication of the vector in the cell independent of the genome.

For integration into the genome of the engineered E. coli strain, the vector may rely on the polynucleotide sequence encoding a polypeptide or any other element of the vector for integration into the genome by homologous or nonhomologous recombination. Alternatively, the vector may contain additional nucleotide sequences for directing integration by homologous recombination into the genome of the engineered E. coli strain at a precise location(s) in the chromosome. To increase the likelihood of integration at a precise location, the integrational elements should preferably contain a sufficient number of nucleic acids, such as 100 to 1,500 base pairs, preferably 400 to 1,500 base pairs, and most preferably 800 to 1,500 base pairs, which have a high degree of identity to the corresponding target sequence to enhance the probability of homologous recombination. The integrational elements may be any sequence that is homologous with the target sequence in the genome of the engineered E. coli strain. Furthermore, the integrational elements may be non-encoding or encoding nucleotide sequences. On the other hand, the vector may be integrated into the genome of the engineered E. coli strain by non-homologous recombination.

For autonomous replication, the vector may further comprise an origin of replication enabling the vector to replicate autonomously in the engineered E. coli strain. The origin of replication may be any plasmid replicator mediating autonomous replication that functions in a cell. The term “origin of replication” or “plasmid replicator” is defined herein as a nucleotide sequence that enables a plasmid or vector to replicate in vivo. Examples of bacterial origins of replication useful in the engineered E. coli strain are the origins of replication of plasmids pBR322, pUC19, pACYC177, and pACYC184 permitting replication in E. coli.

The procedures used to ligate the elements described herein to construct the recombinant expression vectors are well known to one skilled in the art (see, e.g., J. Sambrook, E. F. Fritsch, and T. Maniatus, 1989, Molecular Cloning, A Laboratory Manual, 2d edition, Cold Spring Harbor, New York).

The DNA can also be a control sequence, e.g., promoter, for manipulating the expression of a gene of interest. Non-limiting examples of control sequences are described above.

The DNA can further be a nucleic acid construct for inactivating a gene of interest in an E. coli cell.

The DNA is not to be limited in scope by the specific examples disclosed above, since these examples are intended as illustrations of several embodiments of the disclosure.

Transformation of the DNA into the engineered E. coli strains can be conducted using techniques known in the art, such as electroporation or heat shock as described in the Examples section below.

The transformants described herein can be isolated using standard techniques well-known in the art, including, but not limited to, streak plate isolation, growth in enrichment or selective media, temperature growth selection, filtration, or single cell isolation techniques, such as flow cytometry and microfluidics.

In certain embodiments, the engineered E. coli Nissle strain of the disclosure may comprise a disruption of an endogenous gene. The disruption of the endogenous gene may be carried out using methods well known in the art. A portion of the gene can be disrupted such as the coding region or a control sequence required for expression of the coding region. Such a control sequence of the gene may be a promoter sequence or a functional part thereof, i.e., a part that is sufficient for affecting expression of the gene. For example, a promoter sequence may be inactivated resulting in no expression or a weaker promoter may be substituted for the native promoter sequence to reduce expression of the coding sequence. Other control sequences for possible modification include, but are not limited to, a leader, propeptide sequence, signal sequence, transcription terminator, and transcriptional activator.

Gene deletion techniques to eliminate or reduce expression of a gene may be used with the engineered E. coli Nissle strain of the disclosure. Gene deletion techniques enable the partial or complete removal of the gene thereby eliminating expression. In such methods, deletion of the gene is accomplished by homologous recombination using a plasmid that has been constructed to contiguously contain the 5′ and 3′ regions flanking the gene.

In certain embodiments, one or more (e.g., two, several) nucleotides in a gene or a control sequence thereof required for the transcription or translation thereof are introduced, substituted, and/or removed. For example, nucleotides may be inserted or removed for the introduction of a stop codon, the removal of the start codon, or a frame-shift of the open reading frame. Such a modification may be accomplished by site-directed mutagenesis or PCR generated mutagenesis in accordance with methods known in the art. See, for example, Botstein and Shortle, Science 1985, 229, 4719; Lo et al., Proc. Natl. Acad. Sci. U.S.A. 1985, 81, 2285; Higuchi et al., Nucleic Acids Res 1988, 16, 7351; Shimada, Meth. Mol. Biol. 1996, 57, 157; Ho et al., Gene 1989, 77, 61; Horton et al., Gene 1989, 77, 61; and Sarkar and Sommer, BioTechniques 1990, 8, 404.

Gene disruption techniques by inserting into a gene a disruptive nucleic acid construct comprising a nucleic acid fragment homologous to the gene that will create a duplication of the region of homology and incorporate construct DNA between the duplicated regions may be used. Such a gene disruption can eliminate gene expression if the inserted construct separates the promoter of the gene from the coding region or interrupts the coding sequence such that a non-functional gene product results. A disrupting construct may be simply a selectable marker gene accompanied by 5′ and 3′ regions homologous to the gene. The selectable marker enables identification of transformants containing the disrupted gene.

The process of gene conversion (see, for example, Iglesias and Trautner, Molecular General Genetics 1983, 189, 73-76) may also be used with the engineered E. coli Nissle strain of the disclosure. For example, in the gene conversion method, a nucleotide sequence corresponding to the gene is mutagenized in vitro to produce a defective nucleotide sequence, which is then transformed into the E. coli strain to produce a defective gene. By homologous recombination, the defective nucleotide sequence replaces the endogenous gene. It may be desirable that the defective nucleotide sequence also comprises a marker for selection of transformants containing the defective gene.

Established anti-sense techniques using a nucleotide sequence complementary to the nucleotide sequence of the gene (Parish and Stoker, FEMS Microbiol. Lett. 1997, 154, 151-157) may also be used. More specifically, expression of the gene may be reduced or inactivated by introducing a nucleotide sequence complementary to the nucleotide sequence of the gene, which may be transcribed in the strain and is capable of hybridizing to the mRNA produced in the strain. Under conditions allowing the complementary anti-sense nucleotide sequence to hybridize to the mRNA, the amount of protein translated is thus reduced or eliminated.

Random or specific mutagenesis may also be performed using methods well known in the art, including, but not limited to, chemical mutagenesis (see, for example, Hopwood, The Isolation of Mutants in Methods in Microbiology (J. R. Norris and D. W. Ribbons, eds.) pp. 363-433, Academic Press, New York, 1970). Modification of a gene may be performed by subjecting the engineered E. coli Nissle strain of the disclosure to mutagenesis and screening for strains in which expression of a gene has been reduced or inactivated. The mutagenesis, which may be specific or random, may be performed, for example, by use of a suitable physical or chemical mutagenizing agent, use of a suitable oligonucleotide, or subjecting the DNA sequence to PCR generated mutagenesis. Furthermore, the mutagenesis may be performed by use of any combination of these mutagenizing methods.

Examples of a physical or chemical mutagenizing agent suitable for the present purpose include ultraviolet (UV) irradiation, hydroxylamine, N-methyl-N′-nitro-N-nitrosoguanidine (MNNG), N-methyl-N′-nitrosogaunidine (NTG) O-methyl hydroxylamine, nitrous acid, ethyl methane sulphonate (EMS), sodium bisulphite, formic acid, and nucleotide analogues. When such agents are used, the mutagenesis is typically performed by incubating the strain to be mutagenized in the presence of the mutagenizing agent of choice under suitable conditions, and selecting for mutants exhibiting reduced or no expression of the gene.

In certain embodiments, the modification is unmarked with a selectable marker. Removal of the selectable marker gene may be accomplished by culturing the strain on a counter-selection medium. Where the selectable marker gene contains repeats flanking its 5′ and 3′ ends, the repeats will facilitate the looping out of the selectable marker gene by homologous recombination when the strain is submitted to counter-selection. The selectable marker gene may also be removed by homologous recombination by introducing into the strain a nucleic acid fragment comprising 5′ and 3′ regions of the defective gene, but lacking the selectable marker gene, followed by selecting on the counter-selection medium. By homologous recombination, the defective gene containing the selectable marker gene is replaced with the nucleic acid fragment lacking the selectable marker gene. Other methods known in the art may also be used.

Methods of altering a target nucleic acid sequence within an E. coli cell are also provided. In certain embodiments, the methods comprise providing the cell with a Cas protein. In certain embodiments, the methods comprise providing the cell with a guide RNA. In certain embodiments, the methods comprise providing the cell with a donor nucleic acid sequence. In certain embodiments, the methods comprise providing the cell with a recombinase. In certain embodiments, the E. coli cell is the engineered E. coli Nissle strain deposited under ATCC accession number PTA-______, or a derivative thereof. In certain embodiments, the Cas protein is a Cas9 protein. In certain embodiments, a plurality of guide RNAs that are complementary to different target nucleic acid sequences are provided to the cell and the different target nucleic acid sequences are altered. In certain embodiments, the guide RNA and/or Cas protein are provided on a vector. In certain embodiments, the vector is a plasmid. In certain embodiments, the guide RNA, Cas protein, donor nucleic acid sequence, and/or recombinase are provided on one or more plasmids and provided to the cell by electroporation. In certain embodiments, the donor nucleic acid sequence is introduced into the cell as a single stranded nucleic acid. In certain embodiments, the donor nucleic acid sequence is introduced into the cell as a double stranded nucleic acid. In certain embodiments, the recombinase is a λ-Red recombination system. The terms “λ-Red recombinase”, “λ-Red recombination system”, and “λ-Red system” are used interchangeably to describe a group of enzymes encoded by the bacteriophage λ genes exo, bet, and gam. The enzymes encoded by the three genes work together to increase the rate of homologous recombination in E. coli, which is generally considered to have a relatively low rate of homologous recombination.

The present disclosure provides an engineered E. coli Nissle strain of the disclosure for use as probiotic, in foodstuff, or as a pharmaceutical composition.

A composition that includes the engineered E. coli Nissle strain of the disclosure can include a pharmaceutically acceptable carrier. A pharmaceutically acceptable carrier should be non-toxic to the E. coli and to the subject, and also can optionally include an ingredient that promotes viability of the E. coli during storage. Liquid or gel-based carriers are well known in the art, such as water, fruit juice, glucose or fructose solutions, physiological electrolyte solutions, and glycols such propylene glycol. Carriers also include oleaginous carries such as, for example, white petrolatum, isopropyl myristate, lanolin or lanolin alcohols, mineral oil, fragrant or essential oil, nasturtium extract oil, sorbitan mono-oleate, cetylstearyl alcohol, hydroxypropyl cellulose (MW=100,000 to 1,000,000), or detergents (e.g., polyoxyl stearate or sodium lauryl sulfate). Other suitable carriers include water-in-oil or oil-in-water emulsions and mixtures of emulsifiers and emollients are provided.

A composition that includes the engineered E. coli Nissle strain of the disclosure also can include natural or synthetic flavorings and food-quality coloring agents, thickening agents such as corn starch, guar gum, xanthan gum and the like, binders, disintegrators, coating agents, lubricants, stabilizers, solubilizing agents, suspending agents, excipients, and diluents. Additional components also can be included that, for example, improve palatability, improve shelf-life, and impart nutritional benefits. It would be understood by those in the art that any additional components in a composition must be compatible with maintaining the viability of the engineered E. coli Nissle strain.

In certain embodiments, the composition comprising engineered E. coli Nissle strain is a foodstuff. Foodstuffs include any number of food products that are suitable for human consumption such as, without limitation, milk, yogurt, juices, water, cereals, chewing gum, crackers, candies, cookies, vitamin supplements, meats, and fruits or vegetables (i.e., blended fruits or vegetables such as, e.g., baby food). Foodstuffs also include feed products (e.g., suitable for consumption by livestock or companion animals) including dry animal feeds.

In certain embodiments, the engineered E. coli Nissle strain and a prebiotic are combined prior to administration to produce a symbiotic. Prebiotics are defined in the art as an “ingredient that allows specific changes, both in the composition and/or activity in the gastrointestinal microbiota that confers benefits upon host well-being and health” (see, for example, Roberfroid, 1998, Br. J. Nutr., 80: S197-202). Well characterized prebiotics include, for example, galacto-oligosaccharide (GOS), fructooligosaccharide (FOS), and inulin. GOS and FOS refer to a group of oligomeric, non-digestible carbohydrates that are produced from lactose using beta-galactosidases to catalyze transgalactosylation reactions. These beta-linked glycosides are recalcitrant to digestion by host-secreted enzymes in the small intestine, such that they reach the colon intact and are available to the colonic microbiota. It would be understood by those skilled in the art that other compounds that fall within the definition of a prebiotic also can be used in the compositions and methods described herein.

Administration of a composition that includes the engineered E. coli Nissle strain of the disclosure can be accomplished by any method that delivers at least a portion of the E. coli into the digestive tract of a subject. Therefore, enteral administration is preferred (e.g., orally, sublingually, or rectally), although other routes are not excluded. Generally, the formulation of a composition is dependent upon its intended route of delivery. For example, a composition that includes the engineered E. coli Nissle strain of the disclosure can be formulated as a powder, a granule, a tablet, a capsule, a liquid suspension, a paste, or a syrup.

An effective amount of the engineered E. coli Nissle strain of the disclosure is an amount that achieves a desired result (e.g., treatment or maintenance) in the absence of a toxic, immunological, or allergic reaction in the subject. An effective amount can be at least 10⁴ viable colony forming units per day (CFU/day; e.g., at least 10⁶ CFU/day, 10⁸ to 10¹² CFU/day, or 10¹⁰ CFU/day), which can be administered in a single dose or over multiple doses (e.g., over days, weeks, months or years). When the engineered E. coli Nissle strain of the disclosure is administered over a long period of time (e.g., to maintain a healthy gastrointestinal flora), the effective amount may be less than the foregoing range (e.g., 10 CFU/day to 10³ CFU/day). It would be understood by those in the art that the engineered E. coli Nissle strain of the disclosure can be administered in an amount that exceeds the foregoing range as E. coli Nissle is considered highly safe and has been given GRAS (Generally Recognized as Safe) status by the U.S. Food and Drug Administration (FDA).

Probiotics are reported to produce health benefits which include (1) alleviation of intestinal disorders such as constipation and diarrhea caused by infection by pathogenic organisms, antibiotics, or chemotherapy; (2) stimulation and modulation of the immune system; (3) anti-tumor effects due to inactivation or inhibition of carcinogenic compounds in the gastrointestinal tract by reduction of intestinal bacterial enzyme activities such as beta-glucuronidase, azoreductase, and nitroreductase; (4) reduced production of toxic end products such as ammonia, phenols and other metabolites of protein known to influence liver cirrhosis (5) reduction in serum cholesterol and blood pressure; (6) maintenance of mucosal integrity; (7) alleviation of symptoms of lactose intolerance; (8) prevention of vaginitis. Accordingly, the beneficial effects attributed to probiotics include increased resistance to infectious diseases, healthier immune systems, reduction in irritable bowel syndrome, reductions in blood pressure, reduced serum cholesterol, milder allergies and tumor regression. In animals, for example, probiotics can enhance weight gain or weight loss and improve meat quality, and milk production. Significantly, probiotics can be used to establish and maintain a healthy (e.g., balanced) gastrointestinal flora in an animal and to reduce the effect of gastrointestinal diseases. Gastrointestinal diseases include, without limitation, diarrhea, constipation, loose stool, abdominal inflation, ulcerous colitis, Crohn's disease, irritable bowel syndrome, hypersensitive intestinal syndromes, food toxicity, food allergy, pseudomembranous colitis, hemorrhagic colitis, gastritis, gastroduodenal ulcer, dental caries, and periodontitis. See, for example, Vaughan, Gastrointestinal Microbiology, 2006, CRC Press.

In certain embodiments, the engineered E. coli Nissle strain of the disclosure can be administered with one or more additional probiotic microbial strains. Examples of additional probiotic microorganisms that can be used include yeasts such as Saccharomyces, Candida, Pichia and Torulopsis, moulds such as Aspergillus, Rhizopus, Mucor, and Penicillium and bacteria such as the genera Lactobacillus, Bifidobacterium, Clostridium, Leuconostoc, Bacteroides, Staphylococcus, Lactococcus, Bacillus, Streptococcus, Fusobacterium, Propionibacterium, Enterococcus, Pediococcus, and Micrococcus. Representative examples of additional probiotic microorganisms that can be used include Saccharomyces cereviseae, Bacillus coagulans, Bacillus licheniformis, Bacillus subtilis, Bifidobacterium bifidum, Bifidobacterium infantis, Bifidobacterium longum, Enterococcus faecium, Enterococcus faecalis, Lactobacillus acidophilus, Lactobacillus alimentarius, Lactobacillus casei, Lactobacillus curvatus, Lactobacillus delbruckii, Lactobacillus johnsonii, Lactobacillus farciminus, Lactobacillus gasseri, Lactobacillus helveticus, Lactobacillus rhamnosus, Lactobacillus sake, Lactococcus lactis, Micrococcus varians, Pediococcus acidilactici, and Staphylococcus xylosus.

The engineered E. coli Nissle strain of the disclosure can be provided in an article of manufacture (e.g., in lyophilized form). An article of manufacture also can include one or more pharmaceutically acceptable carriers (e.g., a solvent), and further can include one or more tools for combining and mixing the E. coli with a prebiotic and/or a pharmaceutically acceptable carrier or administering the composition (e.g., a stick or a straw). In addition, an article of manufacture can include one or more other probiotic microorganisms. An article of manufacture also can include appropriate packaging material, and may include written directions or instructions for use (e.g., dosage information) or for administration.

The following numbered embodiments also form part of the present disclosure:
1. An engineered E. coli Nissle strain, or a derivative thereof, having increased transformation efficiency relative to wild type E. coli Nissle.
2. The engineered E. coli Nissle strain of embodiment 1, wherein the strain comprises a genome sequence having at least 90%, at least 95%, at least 98%, at least 99%, at least 99.5% at least 99.6% at least 99.7% at least 99.8%, at least 99.9%, or 100% sequence identity to SEQ ID NO: 2.
3. The engineered E. coli Nissle strain of embodiment 1 or embodiment 2, wherein the strain comprises one or more of the mutations set forth in Table 5.
4. The engineered E. coli Nissle strain of any one of embodiments 1-3, wherein the strain comprises all of the mutations set forth in Table 5.
5. The engineered E. coli Nissle strain of any one of embodiments 1-4, wherein a representative sample of the strain has been deposited under ATCC accession number PTA-______.
6. The engineered E. coli Nissle strain of any one of embodiments 1-5, wherein the derivative comprises a genome sequence having at least 95%, at least 98%, at least 99%, at least 99.5% at least 99.6% at least 99.7% at least 99.8%, or at least 99.9% sequence identity to SEQ ID NO: 2.
7. The engineered E. coli Nissle strain of any one of embodiments 1-6, wherein the derivative comprises all of the mutations set forth in Table 5.
8. The engineered E. coli Nissle strain of any one of embodiments 1-7, wherein the derivative further comprises a heterologous polynucleotide.
9. The engineered E. coli Nissle strain of any one of embodiments 1-8, wherein the derivative further comprises one or more additional mutations.
10. The engineered E. coli Nissle strain of any one of embodiments 1-9, wherein the transformation efficiency is increased at least 2-fold, at least 3-fold, at least 4-fold, at least 5-fold, at least 6-fold, at least 7-fold, at least 8-fold, at least 9-fold, or at least 10-fold relative to the wild type E. coli Nissle strain.
11. A probiotic composition comprising the engineered E. coli Nissle strain of any one of embodiments 1-10.
12. The probiotic composition of embodiment 11, further comprising a prebiotic.
13. The probiotic composition of embodiment 11 or embodiment 12, further comprising pharmaceutically acceptable carrier.
14. The probiotic composition of any one of embodiments 11-13, wherein the composition is formulated as a powder, a granule, a tablet, a capsule, a liquid suspension, a paste, or a syrup.
15. A method for establishing or maintaining a healthy gastrointestinal microbiota in a subject, the method comprising: administering to the subject the engineered E. coli Nissle strain of any one of embodiments 1-10 or the probiotic composition of any one of embodiments 11-13.
16. A method for reducing the effects of a gastrointestinal disorder in a subject, the method comprising: administering to the subject the engineered E. coli Nissle strain of any one of embodiments 1-10 or the probiotic composition of any one of embodiments 11-13.
17. A method for preventing or treating a gastrointestinal disorder in a subject, the method comprising: administering to the subject the engineered E. coli Nissle strain of any one of embodiments 1-10 or the probiotic composition of any one of embodiments 11-13.
18. The method of embodiment 16 or embodiment 17, wherein the composition is administered orally or rectally.
19. The method of any one of embodiments 17-18, wherein the subject is a mammal.
20. A method of obtaining an E. coli transformant, comprising transforming a heterologous polynucleotide into the engineered E. coli Nissle strain of any one of embodiments 1-10.
21 The method of embodiment 20, wherein the transforming comprises electroporation or heat shock.
22. A method of altering a target nucleic acid sequence within an E. coli cell, the method comprising: providing a cell of the engineered E. coli Nissle strain of any one of embodiments 1-10 with a Cas protein, a guide RNA, a donor nucleic acid sequence, and/or a recombinase.
23. The method of embodiment 22, wherein the guide RNA and the Cas protein co-localize at the target nucleic acid sequence, the Cas protein cleaves the target nucleic acid sequence, and the donor nucleic acid sequence is inserted into the target nucleic acid sequence.
24. The method of embodiment 22 or embodiment 23, wherein the recombinase is a 2-Red recombination system.

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

Although the foregoing disclosure has been described in some detail by way of illustration and example for purposes of clarity of understanding, it will be obvious that certain changes and modifications may be practiced within the scope of the appended claims.

The following examples are offered by way of illustration and not by way of limitation.

### EXAMPLES

### Example 1: Adaptive Evolution of E. coli Nissle

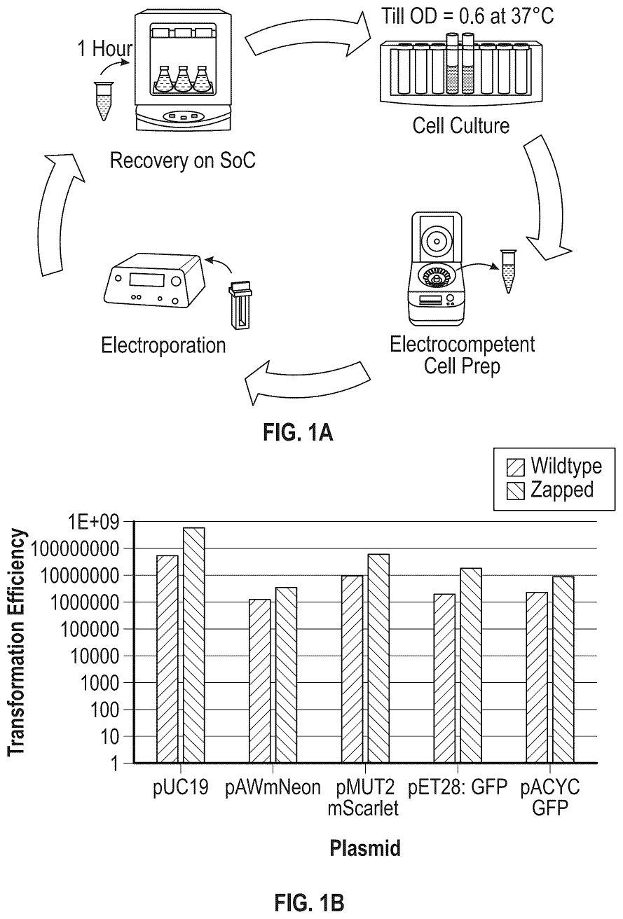

E. coli Nissle was subjected to repeated cycles of electroporation and recovery as schematically represented in FIG. 1A. A 1% inoculum of wild-type EcN was grown up to log phase (OD₆₀₀=0.7) in LB broth at 37° C. The cells were then pelleted using a centrifuge at 1000×g and washed and resuspended in ice-cold water. This was repeated three times to make the cells electrocompetent. 50 μl of these pelleted cells were then mixed with 2 μl of cold plasmid DNA. This mix was pipetted into an electroporation cuvette on ice and left for 20 minutes. The electroporation was carried out using Eppendorf's EPORATOR, H2 ADV PROMO at a potential difference of 1800 V for a set time of five milliseconds and immediately recovered in 950 μl of cold SOC for one hour. The cells were cultured in 5 ml of LB broth overnight at 37° C. This process was repeated several times. A significantly more robust strain was identified at the end of six cycles of electroporation.

### Example 2: Characterization of Engineered Strain of E. coli Nissle

Characterization assays to find transformation efficiency, motility, hydrophobicity, and growth under various conditions of duress were carried out for the engineered strained and compared to wild type EcN. The wildtype strain and the evolved strain were also competed against each other in LB and minimal media. Experiments were also carried out to study the consumption of iron by both strains. To test whether the engineered strain was more resistant to current shock, or was better at accepting plasmid DNA, transformation efficiencies were calculated for the heat shock method as well.

### Transformation Efficiency

The wild-type and engineered strains were grown up to log phase (OD₆₀₀=0.7) and then made electrocompetent by repeated washing with ice-cold water. Plasmid DNA was extracted by miniprepping using the QIAGEN miniprep kit and protocol. 50 μl of the pelleted cells and 2 μl of miniprepped plasmid DNA were mixed in an electroporation cuvette on ice. After 10 minutes of resting the cuvette, the mix was electroporated at 1800 V for five milliseconds using Eppendorf's EPORATOR, H2 ADV PROMO. This was then recovered for one hour in cold SOC broth and serially diluted up to 10⁵ and 10⁶ fold. These dilutions were plated in triplicate on LB agar plates having 1% of the appropriate antibiotic of resistance. These plates were incubated overnight at 37° C. The number of distinct cell colonies was counted using ImageJ. These counts were used to calculate the transformation efficiency using Equation (1).

$$Transformation Efficiency = \frac{No . of colonies}{Dilution \times DNA transformed} \qquad Eq ( 1 )$$

To generalize the trend of transformation efficiency across a variety of plasmids, different origins of replication were used as the exogenous DNA.

The experiments confirmed the improvement of transformation efficiency in the engineered strain consistently across plasmids of various origins of replication (FIG. 1B). Up to a tenfold increase in the transformation efficiency was demonstrated for transformation by electroporation in the engineered strain. TABLE 1 shows transformation efficiency of plasmids of various origins of replication in wild type and engineered strain.

TABLE 1

| | | | | Transformation | Transformation |
|---|---|---|---|---|---|
| | | Antibiotic of | | Efficiency | Efficiency |
| Plasmid | ORI | Resistance | Copy No. | (wild-type) | (Zapped) |
| pUC19 | pMB1 | Ampicillin | 500-700 | 5.47 × 10⁷ | 6.23 × 10⁸ |
| pAWmNeon | pBR322 | Ampicillin | 15-20 | 1.32 × 10⁶ | 3.54 × 10⁶ |
| pMUT2 | ColE2 | Chloramphenicol | 15-20 | 9.5 × 10⁶ | 6.5 × 10⁷ |
| mScarlet | | | | | |
| pACYC | p15A | Chloramphenicol | 10 | 2.4 × 10⁶ | 9.3 × 10⁶ |

### Motility of Cells

The motility of cells was measured using an assay first proposed by Adediran et al. (Infect Immun. 2014 February; 82 (2): 670-82). A 0.3% solution of agar in LB broth was prepared and autoclaved. This mixture was poured into plates and set aside for 40 minutes. Meanwhile, the strains whose motility was being measured were grown up to log phase. 2 μl of the inoculum was pipetted just below the surface of the semisolid gel. These plates were incubated at 37° C. for 8 hours. This experiment was done in triplicate, and the area of the cell swarms at the end of 8 hours was measured using ImageJ.

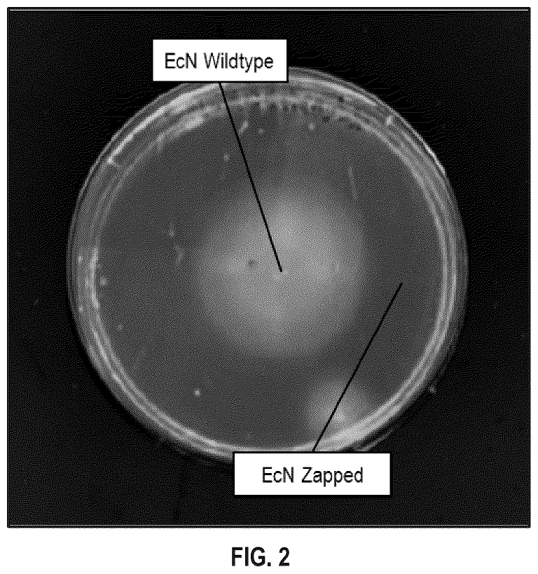

The engineered strain showed a clear and significant increase in motility compared to the wild-type strain. The wildtype EcN was tagged with mNeon Green, while the Zapped strain was tagged with mScarlet. Motility was studied on semi-solid LB Agar. TABLE 2 shows swarm areas measured using the motility assay across triplicate samples of wild-type and Zapped strains. The area of the swarms proved that the engineered strain grew almost five times faster than the wild-type strain. FIG. 2 shows a representation of growth in the semi-solid agar over 24 hours. This was also seen under the microscope, where the engineered EcN cells seemed to be more motile.

TABLE 2

| | Swarm | Swarm | Swarm | Mean Swarm |
|---|---|---|---|---|
| | Area 1 | Area 2 | Area 3 | Area |
| Wild Type | 2.25 cm² | 2.89 cm² | 3.27 cm² | 2.8 cm² |
| Zapped | 8.23 cm² | 8.16 cm² | 14.93 cm² | 10.44 cm² |

### Growth of the Cells Under Different Conditions

The two strains were studied under various conditions of duress, including pH, ethanol concentration, bile salt concentrations, temperature, and osmotic pressure.

To study the growth at various pH levels, cells were grown up to log phase (OD₆₀₀=0.7). A 1% inoculum of these cells in a 96-well plate having LB broth of pH 3, 4, 5, 6, 7 was set up in triplicate. This was put in a plate reader programmed to measure the OD₆₀₀ at an interval of 5 mins for 21 hours. The specific growth constants were calculated using Equation (2).

$$X = {X}_{0} {e}^{μ t} \qquad Eq ( 2 )$$

Similar 96 well plates were set up for an ethanol growth assay having LB of EtOH concentrations between 2% and 25%. Another 96-well plate was set up for LB having bile salt concentration between 0.01% and 0.21. The effect of osmotic pressure was studied using sucrose concentrations from 2.5% to 10%. The growths in all these media were examined by measuring the OD₆₀₀ every 5 minutes for 21 hours. The specific growth constants were calculated.

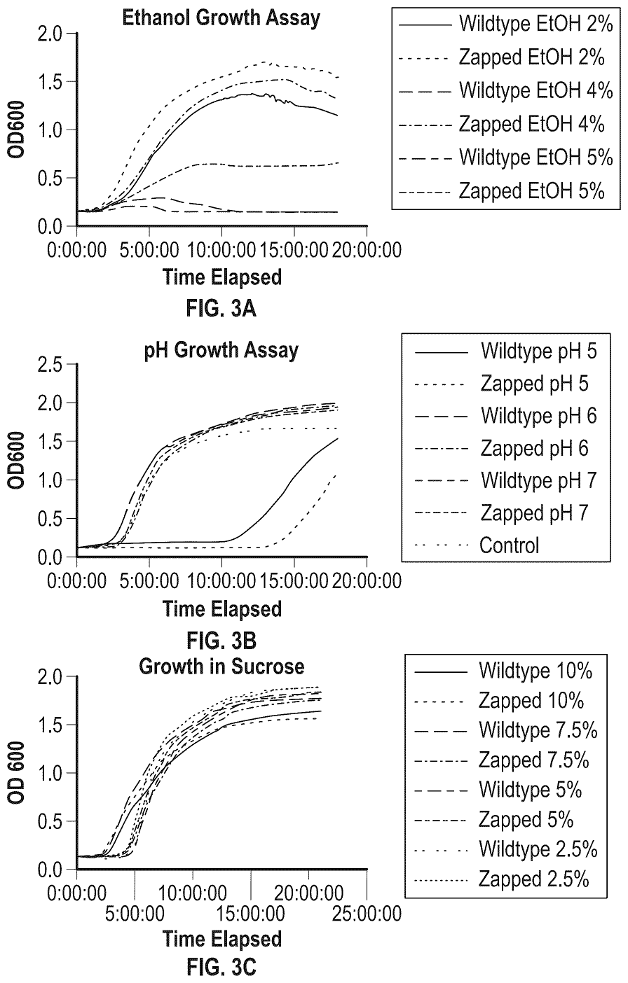

The two strains did not show much difference in growth at 30° C., 37° C., and 42° C. The growth rates in bile salts were also comparable for the two strains. However, engineered strain clearly had a better tolerance to ethanol. The engineered strain tolerated up to 5% EtOH, while wild-type EcN showed stunted growth above 3% EtOH, as shown in FIG. 3A. Below 3%, the growth of both strains was comparable.

The growth of both strains at various levels of pH is shown in FIG. 3B. It was observed that both strains survived in as low as a pH of 5. Both strains had extremely low growth rates at pH 5 but had comparable growths at pH 6 and 7. The growth assay in sucrose revealed good tolerance for both strains at up to 10% concentration of sucrose, as evident from FIG. 3C. The growth rates in EtOH, pH, and sucrose are summarized in TABLE 3.

TABLE 3

| | Growth Rate of Wildtype EcN | Growth Rate of Zapped |
|---|---|---|
| | (hour⁻¹) | EcN (hour⁻¹) |
| Ethanol 2% | 4.979 × 10⁻³ ± 0.0044 | 3.340 × 10⁻³ ± 0.0027 |
| Ethanol 4% | 4.759 × 10⁻⁴ ± 0.0006 | 4.641 × 10⁻³ ± 0.0041 |
| Ethanol 5% | 5.378 × 10⁻⁵ ± 0.00009 | 2.773 × 10⁻³ ± 0.0025 |
| pH 5 | 3.035 × 10⁻² ± 0.0231 | 3.042 × 10⁻² ± 0.0261 |
| pH 6 | 5.496 × 10⁻² ± 0.0276 | 6.753 × 10⁻² ± 0.0219 |
| PH 7 | 5.316 × 10⁻² ± 0.0314 | 6.989 × 10⁻² ± 0.0249 |
| Sucrose 10% | 4.123 × 10⁻² ± 0.0207 | 4.051 × 10⁻² ± 0.0278 |
| Sucrose 7.5% | 4.527 × 10⁻² ± 0.0179 | 5.069 × 10⁻² ± 0.0278 |
| Sucrose 5% | 5.082 × 10⁻² ± 0.0278 | 5.542 × 10⁻² ± 0.0223 |
| Sucrose 2.5% | 5.534 × 10⁻² ± 0.0289 | 5.966 × 10⁻² ± 0.0213 |

### Hydrophobicity

Hydrophobicity was measured using a standard partitioning assay. Fresh cultures of both strains were grown till log phase (OD₆₀₀=0.7). The OD was found using Thermo Fischer's NanoDrop^{C}. The OD₅₄₆ of the cell (OD₁) was adjusted to 0.3 using 10 mM CaCO₃ solution. 4 ml of the cell suspension was transferred to individual test tubes having 1 ml of dodecane and vortexed at 2500 rpm for 10 minutes to homogenize the phases. The phases were allowed to separate for 15 minutes. The OD₅₄₆ of the aqueous phase (OD₂) was measured.

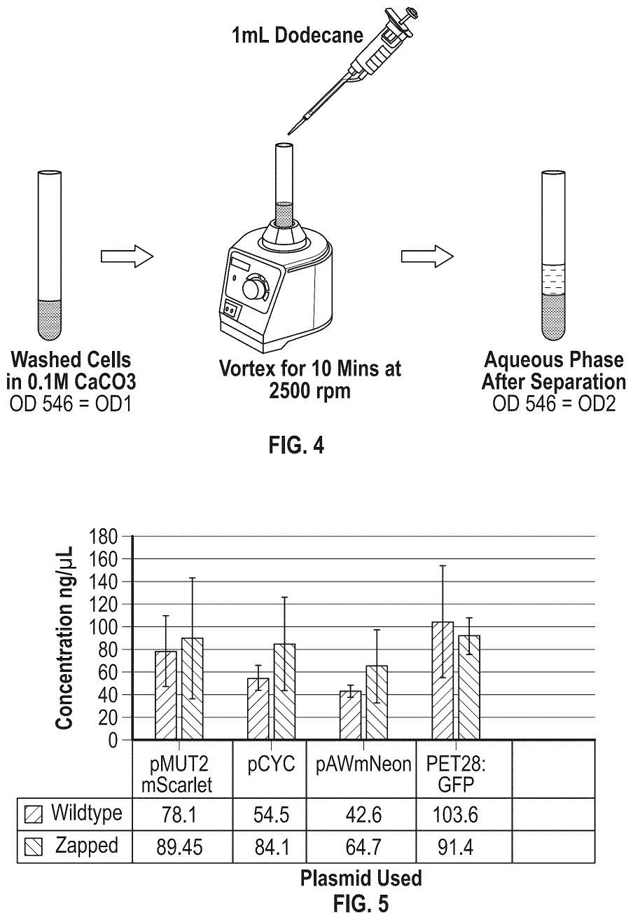

The mean percentage of partitioning was calculated using triplicate samples. A schematic representation of the protocol is shown in FIG. 4. Partitioning of the bacteria suspension is calculated using Equation (3).

$$Percentage of Partitioning = \frac{OD 1 - OD 2}{OD 1} \times 100 \qquad Eq ( 3 )$$

The assay for membrane hydrophobicity demonstrated significant difference in phase partitioning, as shown in TABLE 4. The data shows that the wild-type strain of EcN is hydrophilic, while the engineered strain is hydrophobic.

TABLE 4

| | Wild-type | | | Zapped | | |
|---|---|---|---|---|---|---|
| OD1 | 0.29 | 0.27 | 0.27 | 0.26 | 0.27 | 0.25 |
| OD2 | 0.14 | 0.17 | 0.14 | 0.12 | 0.12 | 0.11 |
| Percentage of | 51.724 | 37.037 | 48.148 | 53.846 | 55.556 | 56.000 |
| Partitioning | | | | | | |
| Average | 45.636 | | | 55.134 | | |
| Partitioning | | | | | | |

### Plasmid Concentration

To examine whether the difference was simply in the amount of plasmid that was being made by the two cell lines, the cells were cultured, and plasmids were extracted in biological sextuplets. A nanodrop was then used to find the concentration of DNA in the minipreps. This was repeated for several ORIs and is shown in FIG. 5.

### Competition Assay

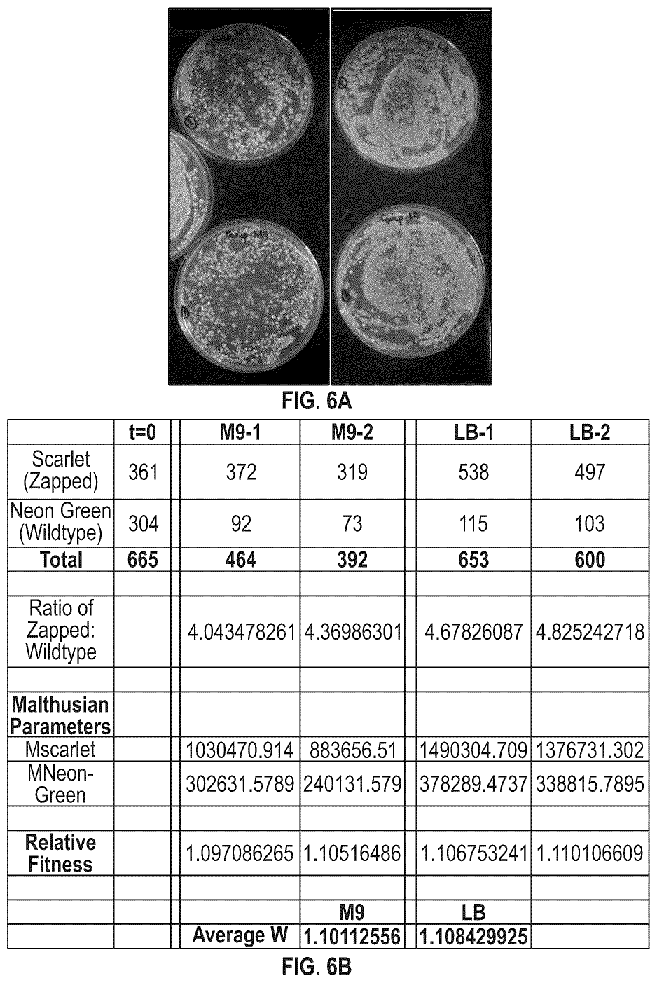

Next, how these two strains compete in the same environment was studied. To compete the two strains, they were transformed with plasmids containing fluorescent markers. The wildtype expressed mNeon Green while the engineered strain expressed mScarlet constitutively. Relative fitness was calculated as per the Malthusian Population Theory. The competition assay revealed the engineered strain as having about 1.1 times the relative fitness of the wildtype strain in both LB and minimal media (FIG. 6A-B).

### Iron Consumption

An assay to measure consumption of iron in E. coli was first demonstrated by Yeowell et al (Antimicrob Agents Chemother. 1982 December; 22 (6): 961-8). The consumption of iron by the two strains of EcN was compared by finding the lethality, or minimum inhibition concentration of Streptonigrin for both the strains. The MIC was measured in LB with 50 μM Ammonium Ferrous Sulphate. Streptonigrin was dissolved in DMSO. The iron source was 3 μM Ferric Chloride with 1 mM sodium ascorbate. A 96 well plate was set up with LB having streptonigrin in varying concentrations from 0.1 μg/ml to 10 μg/ml. 1% of the iron source was supplemented. A 1% inoculum of the EcN was mixed into the wells and set into the plate reader at 37° C. Readings were taken down at a 10-minute interval. Additionally, growth assays in M63 media and M9 media supplemented with no iron, and 0.4% iron were carried out in similar 96 well plates with similar parameters.

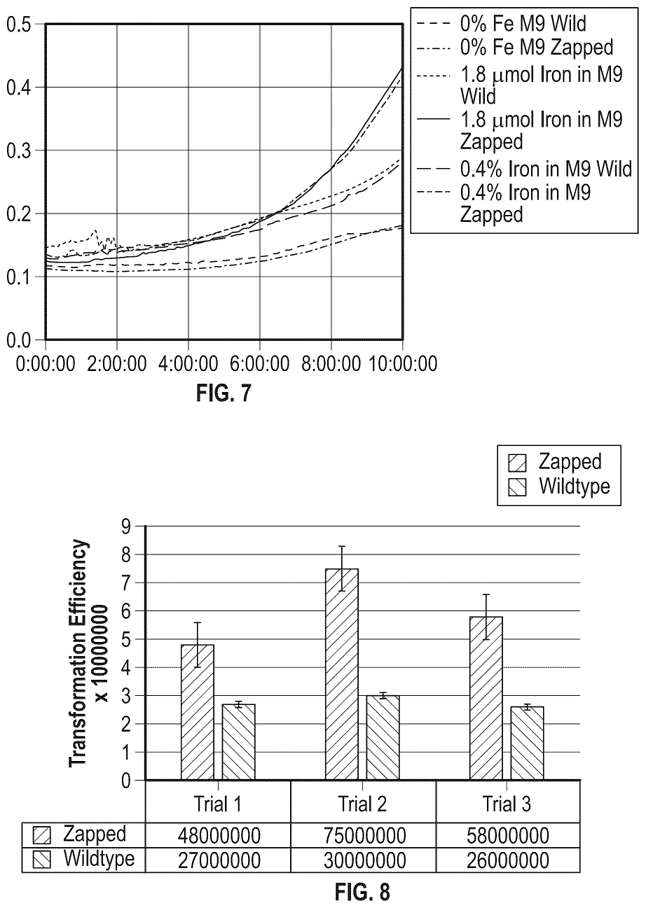

The growth of the two strains in M9 media was comparable. However, as the amount of Iron increased, the growth of the Zapped strain exceeded the wildtype as shown in FIG. 7.

### Heat Shock Transformation

A 1% inoculum of wild-type and Zapped EcN were grown up to log phase (OD₆₀₀=0.7) in LB broth at 37° C. These were pelleted in the centrifuge at 10000×g. The cell pellets were washed and resuspended in ice-cold 0.1M CaCl₂) solution. This was followed by centrifugation to pellet the cells again. The washing and spinning down was repeated three times. The final mixture was resuspended in 1 ml of 0.1 CaCl₂) solution and placed on ice for 20 mins. 2 μL of pUC 19 DNA was placed on ice in a centrifuge tube and then mixed gently with 250 μL of the EcN cells. This mixture was left on ice for 30 mins. Then the centrifuge tube was placed in a heat bath at 42° C. for 40 seconds and placed back onto ice for 10 minutes. This was recovered in 250 μL of SOC at 37° C. for an hour and then plated in dilutions.

The transformation efficiencies were calculated for heat-shock transformation for both the strains using pUC19 as plasmid DNA. There was an average of two-fold increase in the transformation in the Zapped strain (FIG. 8).

### Example 3: Whole Genome Sequencing

Further to identify the specific mutations that were enabled by the ALE, genome sequencing of the engineered and wild type strain was done. Whole Genome Sequencing of the wild-type and the engineered strain were carried out at Azenta Life Sciences. DNA was extracted from the two strains and PacBio whole genome sequencing was carried out. The isolated DNA was then prepared into a sequencing library by adding hairpin adapters to each end of the DNA fragments. This library was then loaded onto the PacBio instrument. The emitted light signals were recorded. Real-time data collection was followed by base calling and bioinformatics analysis.

CCS reads were aligned to the provided reference genome using the PacBio software pbmm2 1.2.0. Variants were called using Sentieon 202110.01 (DNAScope algorithm) and normalized (left alignment of INDELs and splitting multiallelic sites into multiple sites) using bcftools 1.13. Variants were then filtered based on allele frequency.

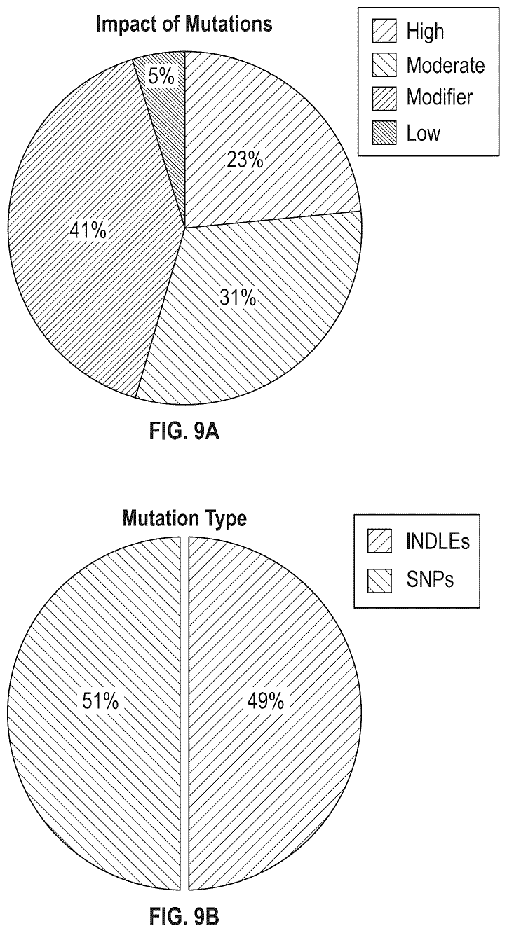

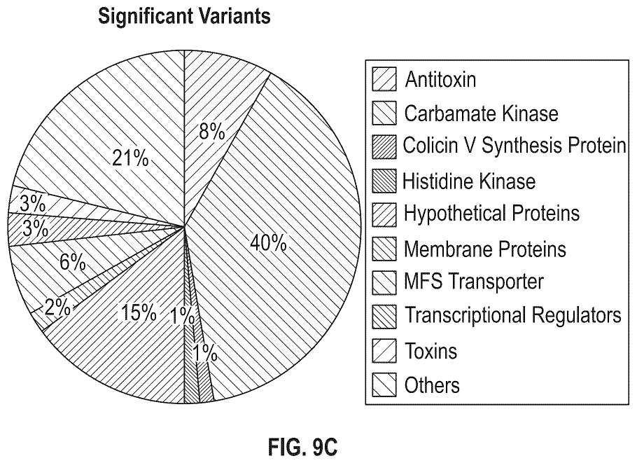

The sequence assembly was screened for INDEL and SNP variants. A total of 292 variants were identified across the two strains (TABLE 5). This included 144 INDEL variants and 148 SNP variations. Out of these, 69 were high-impact mutations, 90 were of moderate impact, and 14 were of low impact. The rest of the mutations were modifier mutations. FIG. 9A-C shows key results of the sequencing.

Several mutations were identified in the Carbamate Kinase gene (ArcC). This is known to be a catalyst in carbamoyl phosphate synthesis. Multiple mutations were also seen in the MES transporter. The major facilitator superfamily (MFS) is one of the two largest families of membrane transporters. The CadB gene also showed several mutations. CadB has been known to help with tolerance of E. coli in acidic conditions. The engineered strained showed better tolerance to pH, which may be explained by these mutations in CadB.

A total of five transcriptional regulators were found to be affected by the mutations including transcriptional regulators in the Fis, TorR, and RhaR Families. Fis is a global transcriptional regulator modulating the expression of over 13% genes in the bacterial genome. Fis, or “factor for inversion stimulation”, is a DNA-binding protein with a primary role in organizing and maintaining nucleoid structure by influencing the production of gyrase and topoisomerase-I. It plays a role in various cellular processes, including transcription, chromosomal replication, DNA inversion, phage integration/excision, and DNA transposition. Furthermore, Fis can create topological barriers, impeding supercoiling diffusion around specific promoters.

TorR is a transcriptional DNA-binding regulator with both positive and negative roles. It positively regulates genes associated with TMAO induction and tryptophan metabolism while negatively regulating glutamate decarboxylase genes and its own expression. TorR is also involved in anaerobic regulation and plays a part in alkaline and acid stress responses.

RhaR is yet another transcriptional regulator that featured in the variant analysis. It is an essential part of the RhaBAD regulon, and plays a crucial role in regulating the expression of genes involved in the utilization of rhamnose as a carbon source. RhaR induces RhaS in the presence of rhamnose. RhaR is a member of the AraC family of transcriptional regulators.

TABLE 5

| Position | | | | | |
|---|---|---|---|---|---|
| (relative | | | | | |
| to SEQ | | | | Predicted | |
| ID NO: 1) | Ref | Alt | Type | Impact | Gene |
| 257307 | TG | T | INDEL | HIGH | ECOLIN_01245 |
| 261611 | C | CA | INDEL | MODIFIER | ECOLIN_01235 |
| 287006 | CG | C | INDEL | HIGH | ECOLIN_01465 |
| 313346 | TG | T | INDEL | MODIFIER | ECOLIN_01570 |
| 372997 | AC | A | INDEL | HIGH | ECOLIN_01800 |
| 547536 | TC | T | INDEL | HIGH | ECOLIN_02590 |
| 577005 | CA | C | INDEL | MODIFIER | ECOLIN_02735 |
| 619073 | CT | C | INDEL | HIGH | ECOLIN_02915 |
| 905646 | TC | T | INDEL | HIGH | ECOLIN_04325 |
| 948388 | TC | T | INDEL | MODIFIER | ECOLIN_04550 |
| 1100140 | TC | T | INDEL | MODIFIER | ECOLIN_05190 |
| 1106624 | TG | T | INDEL | HIGH | ECOLIN_05225 |
| 1155295 | CG | C | INDEL | HIGH | ECOLIN_05515 |
| 1155975 | TG | T | INDEL | HIGH | ECOLIN_05515 |
| 1207958 | TG | T | INDEL | MODIFIER | ECOLIN_05765 |
| 1214983 | AG | A | INDEL | HIGH | ECOLIN_05835 |
| 1217272 | AG | A | INDEL | HIGH | ECOLIN_05860 |
| 1330104 | A | ATTC | INDEL | MODERATE | ECOLIN_06500 |
| 1330105 | A | AGTAAACCAC | INDEL | HIGH | ECOLIN_06500 |
| | | (SEQ ID NO: 3) | | | |
| 1431503 | G | T | SNP | MODERATE | ECOLIN_07110 |
| 1511516 | TA | T | INDEL | MODIFIER | ECOLIN_07475 |
| 1559408 | TC | T | INDEL | HIGH | ECOLIN_07715 |
| 1642042 | TG | T | INDEL | HIGH | ECOLIN_08080 |
| 1693592 | G | GT | INDEL | HIGH | ECOLIN_08295 |
| 1738308 | TC | T | INDEL | MODIFIER | ECOLIN_08490 |
| 1775453 | T | TA | INDEL | MODIFIER | ECOLIN_08670 |
| 1797994 | TC | T | INDEL | HIGH | ECOLIN_08785 |
| 1802903 | C | CT | INDEL | MODIFIER | ECOLIN_08815 |
| 1833436 | TC | T | INDEL | HIGH | ECOLIN_09000 |
| 1908015 | C | CA | INDEL | MODIFIER | ECOLIN_09345 |
| 1946344 | TC | T | INDEL | HIGH | ECOLIN_09555 |
| 1954709 | TC | T | INDEL | HIGH | ECOLIN_09600 |
| 1972000 | C | CA | INDEL | MODIFIER | ECOLIN_09660 |
| 2029609 | G | GT | INDEL | HIGH | ECOLIN_09995 |
| 2072645 | G | GT | INDEL | MODIFIER | ECOLIN_10230 |
| 2150037 | CG | C | INDEL | HIGH | ECOLIN_10720 |
| 2180269 | CT | C | INDEL | HIGH | ECOLIN_10880 |
| 2188100 | CT | C | INDEL | HIGH | ECOLIN_10920 |
| 2203779 | TC | T | INDEL | MODIFIER | ECOLIN_10995 |
| 2222419 | TG | T | INDEL | HIGH | ECOLIN_11035 |
| 2231010 | TC | T | INDEL | HIGH | ECOLIN_11065 |
| 2275579 | TG | T | INDEL | HIGH | ECOLIN_11170 |
| 2290073 | AT | A | INDEL | HIGH | ECOLIN_11200 |
| 2338206 | TC | T | INDEL | MODIFIER | ECOLIN_11435 |
| 2343227 | CA | C | INDEL | MODIFIER | ECOLIN_11470 |
| 2370965 | TC | T | INDEL | MODIFIER | ECOLIN_11585 |
| 2469948 | TC | T | INDEL | HIGH | ECOLIN_12000 |
| 2472325 | TC | T | INDEL | HIGH | ECOLIN_12005 |
| 2534241 | TC | T | INDEL | HIGH | ECOLIN_12290 |
| 2673940 | TC | T | INDEL | HIGH | ECOLIN_12890 |
| 2722786 | TC | T | INDEL | MODIFIER | ECOLIN_13120 |
| 2723200 | TC | T | INDEL | MODIFIER | ECOLIN_13120 |
| 2727344 | TC | T | INDEL | MODIFIER | ECOLIN_13135 |
| 2740342 | TC | T | INDEL | HIGH | ECOLIN_13170 |
| 2823249 | TG | T | INDEL | HIGH | ECOLIN_13585 |
| 2825576 | GA | G | INDEL | HIGH | ECOLIN_13590 |
| 2925375 | TC | T | INDEL | HIGH | ECOLIN_13975 |
| 2984481 | C | CA | INDEL | HIGH | ECOLIN_14250 |
| 3091331 | GC | G | INDEL | MODIFIER | ECOLIN_14825 |
| 3109405 | TC | T | INDEL | MODIFIER | ECOLIN_14925 |
| 3131290 | TC | T | INDEL | HIGH | ECOLIN_15025 |
| 3190080 | TC | T | INDEL | HIGH | ECOLIN_15290 |
| 3234031 | TG | T | INDEL | HIGH | ECOLIN_15460 |
| 3290228 | CG | C | INDEL | MODIFIER | ECOLIN_15675 |
| 3318100 | CA | C | INDEL | HIGH | ECOLIN_15840 |
| 3381051 | TC | T | INDEL | HIGH | ECOLIN_16160 |
| 3394188 | TC | T | INDEL | MODIFIER | ECOLIN_16210 |
| 3408230 | C | G | SNP | MODERATE | ECOLIN_16295 |
| 3408232 | G | A | SNP | MODERATE | ECOLIN_16295 |
| 3408236 | G | A | SNP | MODERATE | ECOLIN_16295 |
| 3408254 | C | T | SNP | MODERATE | ECOLIN_16295 |
| 3408260 | C | G | SNP | MODERATE | ECOLIN_16295 |
| 3408264 | A | T | SNP | LOW | ECOLIN_16295 |
| 3408265 | G | C | SNP | MODERATE | ECOLIN_16295 |
| 3408271 | G | A | SNP | MODERATE | ECOLIN_16295 |
| 3408282 | A | C | SNP | LOW | ECOLIN_16295 |
| 3408298 | CACT | C | INDEL | MODERATE | ECOLIN_16295 |
| 3408302 | GGCA | G | INDEL | MODERATE | ECOLIN_16295 |
| 3408310 | C | A | SNP | MODERATE | ECOLIN_16295 |
| 3408312 | ACCACC | A | INDEL | HIGH | ECOLIN_16295 |
| 3408319 | C | G | SNP | MODERATE | ECOLIN_16295 |
| 3408320 | A | AAAAAT | INDEL | HIGH | ECOLIN_16295 |
| 3408324 | C | CTCT | INDEL | MODIFIER | ECOLIN_16300 |
| 3408327 | C | A | SNP | MODIFIER | ECOLIN_16300 |
| 3408332 | A | AGCCT | INDEL | MODIFIER | ECOLIN_16300 |
| 3408334 | C | CTGCTACGGCCTGGTGTTT | INDEL | MODIFIER | ECOLIN_16300 |
| | | (SEQ ID NO: 4) | | | |
| 3408339 | C | CACGCCACTTTTCCATT | INDEL | MODIFIER | ECOLIN_16300 |
| | | (SEQ ID NO: 5) | | | |
| 3408343 | A | ATATCT | INDEL | MODIFIER | ECOLIN_16300 |
| 3408346 | AC | A | INDEL | MODIFIER | ECOLIN_16300 |
| 3408350 | C | T | SNP | MODIFIER | ECOLIN_16300 |
| 3408353 | GTTACA | G | INDEL | MODIFIER | ECOLIN_16300 |
| 3408361 | G | A | SNP | MODIFIER | ECOLIN_16300 |
| 3408363 | A | AT | INDEL | MODIFIER | ECOLIN_16300 |
| 3408367 | A | C | SNP | MODIFIER | ECOLIN_16300 |
| 3408369 | G | GT | INDEL | MODIFIER | ECOLIN_16300 |
| 3408384 | T | C | SNP | MODERATE | ECOLIN_16300 |
| 3408401 | A | C | SNP | LOW | ECOLIN_16300 |
| 3408457 | T | C | SNP | LOW | ECOLIN_16300 |
| 3408468 | T | C | SNP | MODERATE | ECOLIN_16300 |
| 3408471 | C | T | SNP | MODERATE | ECOLIN_16300 |
| 3408480 | A | G | SNP | MODERATE | ECOLIN_16300 |
| 3410228 | G | A | SNP | MODERATE | ECOLIN_16315 |
| 3410238 | A | G | SNP | LOW | ECOLIN_16315 |
| 3410240 | T | C | SNP | MODERATE | ECOLIN_16315 |
| 3410243 | G | A | SNP | HIGH | ECOLIN_16315 |
| 3410249 | C | G | SNP | MODERATE | ECOLIN_16315 |
| 3413110 | TG | T | INDEL | HIGH | ECOLIN_16345 |
| 3451296 | G | GT | INDEL | HIGH | ECOLIN_16530 |
| 3520054 | TC | T | INDEL | HIGH | ECOLIN_16875 |
| 3554413 | CA | C | INDEL | HIGH | ECOLIN_17025 |
| 3604748 | AG | A | INDEL | MODIFIER | ECOLIN_17265 |
| 3640318 | TC | T | INDEL | MODIFIER | ECOLIN_17435 |
| 3764568 | TC | T | INDEL | HIGH | ECOLIN_18045 |
| 3844856 | TC | T | INDEL | HIGH | ECOLIN_18570 |
| 3873401 | CG | C | INDEL | HIGH | ECOLIN_18720 |
| 3888054 | G | T | SNP | MODIFIER | ECOLIN_18785 |
| 4048903 | TC | T | INDEL | HIGH | ECOLIN_19485 |
| 4175223 | AG | A | INDEL | HIGH | ECOLIN_20030 |
| 4208844 | AG | A | INDEL | MODIFIER | ECOLIN_20180 |
| 4208848 | A | G | SNP | MODIFIER | ECOLIN_20180 |
| 4208851 | T | C | SNP | MODIFIER | ECOLIN_20180 |
| 4265268 | CG | C | INDEL | HIGH | ECOLIN_20530 |
| 4341919 | TC | T | INDEL | MODIFIER | ECOLIN_20865 |
| 4383425 | G | GT | INDEL | MODIFIER | ECOLIN_21080 |
| 4404105 | TA | T | INDEL | MODIFIER | ECOLIN_21155 |
| 4463623 | T | TGAGC | INDEL | MODIFIER | ECOLIN_21485 |
| 4463628 | A | AC | INDEL | MODIFIER | ECOLIN_21485 |
| 4463630 | G | GCAT | INDEL | MODIFIER | ECOLIN_21485 |
| 4463634 | A | C | SNP | MODIFIER | ECOLIN_21485 |
| 4463637 | T | G | SNP | MODIFIER | ECOLIN_21485 |
| 4463638 | T | TCGG | INDEL | MODIFIER | ECOLIN_21485 |
| 4463640 | CA | C | INDEL | MODIFIER | ECOLIN_21485 |
| 4463644 | G | C | SNP | MODIFIER | ECOLIN_21485 |
| 4463645 | C | T | SNP | MODIFIER | ECOLIN_21485 |
| 4463650 | A | AAAGTACCTGCATT | INDEL | MODIFIER | ECOLIN_21485 |
| | | (SEQ ID NO: 6) | | | |
| 4463653 | G | GAGA | INDEL | MODIFIER | ECOLIN_21485 |
| 4463655 | T | TCA | INDEL | MODIFIER | ECOLIN_21485 |
| 4463658 | T | TCGGCCAGG | INDEL | MODIFIER | ECOLIN_21485 |
| 4463660 | T | C | SNP | MODIFIER | ECOLIN_21485 |
| 4463664 | GAAAA | G | INDEL | MODIFIER | ECOLIN_21485 |
| 4463678 | A | G | SNP | MODIFIER | ECOLIN_21485 |
| 4463679 | A | AGC | INDEL | MODIFIER | ECOLIN_21485 |
| 4463692 | T | TCCACCATGTTTGAGTTT | INDEL | HIGH | ECOLIN_21485 |
| | | GTTCCGGAAAGTCTTTCC | | | |
| | | GGGTC | | | |
| | | (SEQ ID NO: 7) | | | |
| 4463745 | A | G | SNP | LOW | ECOLIN_21485 |
| 4463757 | G | A | SNP | LOW | ECOLIN_21485 |
| 4609507 | A | ACCGTGTACGTACAAGCA | INDEL | MODIFIER | ECOLIN_22180 |
| | | GTGGGAGCCTCTTAATGG | | | |
| | | GGTGACTGCGTACCTTTT | | | |
| | | GTATAATGGGTCAGCGAC | | | |
| | | TTATATTCTGTAGCAAGG | | | |
| | | TTAACCGAATAGGGGAGC | | | |
| | | CGAAGGGAAACCGAGTCT | | | |
| | | TAACTGGGCGTTAAGTTG | | | |
| | | CAGGGTATAGACCCGAAA | | | |
| | | CCCGGTGATCTAGCCATG | | | |
| | | GGCAGGTTGAAGGTTGGG | | | |
| | | TAACACTAACTGGAGGAC | | | |
| | | CGAACCGACTAATGTTGA | | | |
| | | AAAATTAGCGGATGACTT | | | |
| | | GTGGCTGGGGGTGAAAGG | | | |
| | | CCAATCAAACCGGGAGAT | | | |
| | | AGCTGGTTCTCCCCGAAA | | | |
| | | GCTATTTAGGTAGCGCCT | | | |
| | | CGTGAATTCATCTCCGGG | | | |
| | | GGTAGAGCACTGTTTCGG | | | |
| | | CAAGGGGGTCATCCCGAC | | | |
| | | TTACCAACCCGATGCAAA | | | |
| | | CTGCGAATACCGGAGAAT | | | |
| | | GTTATCACGGGAGACACA | | | |
| | | CGGCGGGTGCTAACGTCC | | | |
| | | GTCGTGAAGAGGGAAACA | | | |
| | | ACCCAGACCGCCAGCTAA | | | |
| | | GGTCCCAAAGTCATGGTT | | | |
| | | AAGTGGGAAACGATGTGG | | | |
| | | GAAGGCCCAGACAGCCAG | | | |
| | | GATGTTGGCTTAGAAGCA | | | |
| | | GCCATCATTTAAAGAAAG | | | |
| | | CGTAATAGCTCACTGGTC | | | |
| | | GAGTCGGCCTGCGCGGAA | | | |
| | | GATGTAACGGGGCTAAAC | | | |
| | | CATGCACCGAAGCTGCGG | | | |
| | | CAGCGACACTATGTGTTG | | | |
| | | TTGGGTAGGGGAGCGTTC | | | |
| | | TGTAAGCCTGTGAAGGTG | | | |
| | | GCCTGTGAGGGTTGCTGG | | | |
| | | AGGTATCAGAAGTGCGAA | | | |
| | | TGCTGACATAAGTAACGA | | | |
| | | TAAAGCGGGTGAAAAGCC | | | |
| | | CGCTCGCCGGAAGACCAA | | | |
| | | GGGTTCCTGTCCAACGTT | | | |
| | | AATCGGGGCAGGGTGAGT | | | |
| | | CGACCCCTAAGGCGAGGC | | | |
| | | CGAAAGGCGTAGTCGATG | | | |
| | | GGAAACAGGTTAATATTC | | | |
| | | CTGTACTTGGTGTTACTG | | | |
| | | CGAAGGGGGGACGGAGAA | | | |
| | | GGCTATGTTGGCCGGGCG | | | |
| | | ACGGTTGTCCCGGTTTAA | | | |
| | | GCGTGTAGGCTGGTTTTC | | | |
| | | CAGGCAAATCCGGAAAAC | | | |
| | | CAAGGCTGAGGCGTGATG | | | |
| | | ACGAGGCACTACGGTGCT | | | |
| | | GAAGCGACAAATGCCCTG | | | |
| | | CTTCCAGGAAAAGCCTCT | | | |
| | | AAGCATCAGGTAACATCA | | | |
| | | AATCGTACCCCAAACCGA | | | |
| | | CACAGGTGGTCAGGTAGA | | | |
| | | GAATACCAAGGCGCTTGA | | | |
| | | GAGAACTCGGGTGAAGGA | | | |
| | | ACTAGGCAAAATGGTGCC | | | |
| | | GTAACTTCGGGAGAAGGC | | | |
| | | ACGCTGATATGTAGGTGA | | | |
| | | AGCGACTTGCTCGTGGAG | | | |
| | | CTGAAATCAGTCGAAGAT | | | |
| | | ACCAGCTGGCTGCAACTG | | | |
| | | TTTATTAAAAACACAGCA | | | |
| | | CTGTGCAAACACGAAAGT | | | |
| | | GGACGTATACGGTGTGAC | | | |
| | | GCCTGCCCGGTGCCGGAA | | | |
| | | GGTTAATTGATGGGGTTA | | | |
| | | GCGGTAACGCGAAGCTCT | | | |
| | | TGATCGAAGCCCCGGTAA | | | |
| | | ACGGCGGCCGTAACTATA | | | |
| | | ACGGTCCTAAGGTAGCGA | | | |
| | | AATTCCTTGTCGGGTAAG | | | |
| | | TTCCGACCTGCACGAATG | | | |
| | | GCGTAATGATGGCCAGGC | | | |
| | | TGTCTCCACCCGAGACTC | | | |
| | | AGTGAAATTGAACTCGCT | | | |
| | | GTGAAGATGCAGTGTACC | | | |
| | | CGCGGCAAGACGGAAAGA | | | |
| | | CCCCGTGAACCTTTACTA | | | |
| | | TAGCTTGACACTGAACAT | | | |
| | | TGAGCCTTGATGTGTAGG | | | |
| | | ATAGGTGGGAGGCTTTGA | | | |
| | | AGTGTGGACGCCAGTCTG | | | |
| | | CATGGAGCCGACCTTGAA | | | |
| | | ATACCACCCTTTAATGTT | | | |
| | | TGATGTTCTAACGTTGGC | | | |
| | | CCGTAATCCGGGTTGCGG | | | |
| | | ACAGTGTCTGGTGGGTAG | | | |
| | | TTTGACTGGGGCGGTCTC | | | |
| | | CTCCTAAAGAGTAACGGA | | | |
| | | GGAGCACGAAGGTTGGCT | | | |
| | | AATCCTGGTCGGACATCA | | | |
| | | GGAGGTTAGTGCAATGGC | | | |
| | | ATAAGCCAGCTTGACTGC | | | |
| | | GAGCGTGACGGCGCGAGC | | | |
| | | AGGTGCGAAAGCAGGTCA | | | |
| | | TAGTGATCCGGTGGTTCT | | | |
| | | GAATGGAAGGGCCATCGC | | | |
| | | TCAACGGATAAAAGGTAC | | | |
| | | TCCGGGGATAACAGGCTG | | | |
| | | ATACCGCCCAAGAGTTCA | | | |
| | | TATCGACGGCGGTGTTTG | | | |
| | | GCACCTCGATGTCGGCTC | | | |
| | | ATCACATCCTGGGGCTGA | | | |
| | | AGTAGGTCCCAAGGGTAT | | | |
| | | GGCTGTTCGCCATTTAAA | | | |
| | | GTGGTACGCGAGCTGGGT | | | |
| | | TTAGAACGT | | | |
| | | (SEQ ID NO: 8) | | | |
| 4610114 | TC | T | INDEL | MODIFIER | ECOLIN_22185 |
| 4649091 | TC | T | INDEL | MODIFIER | ECOLIN_22350 |
| 4709868 | C | CA | INDEL | HIGH | ECOLIN_22645 |
| 4729061 | TC | T | INDEL | MODIFIER | ECOLIN_22710 |
| 4753558 | TG | T | INDEL | HIGH | ECOLIN_22830 |
| 4756638 | C | T | SNP | MODIFIER | ECOLIN_22825 |
| 4826691 | G | A | SNP | MODIFIER | ECOLIN_23160 |
| 4826713 | A | T | SNP | MODIFIER | ECOLIN_23160 |
| 4826728 | G | T | SNP | MODIFIER | ECOLIN_23160 |
| 4826746 | C | T | SNP | MODIFIER | ECOLIN_23160 |
| 4826749 | T | C | SNP | MODIFIER | ECOLIN_23160 |
| 4826752 | T | C | SNP | MODIFIER | ECOLIN_23160 |
| 4827732 | AGT | A | INDEL | MODIFIER | ECOLIN_23165 |
| 4827736 | T | TCG | INDEL | MODIFIER | ECOLIN_23165 |
| 4827746 | G | T | SNP | MODIFIER | ECOLIN_23165 |
| 4827747 | A | C | SNP | MODIFIER | ECOLIN_23165 |
| 4827750 | T | C | SNP | MODIFIER | ECOLIN_23165 |
| 4827753 | C | T | SNP | MODIFIER | ECOLIN_23165 |
| 4827763 | G | A | SNP | MODIFIER | ECOLIN_23165 |
| 4827766 | G | A | SNP | MODIFIER | ECOLIN_23165 |
| 4827770 | C | T | SNP | MODIFIER | ECOLIN_23165 |
| 4827772 | G | A | SNP | MODIFIER | ECOLIN_23165 |
| 4827774 | A | T | SNP | MODIFIER | ECOLIN_23165 |
| 4827776 | A | T | SNP | MODIFIER | ECOLIN_23165 |
| 4827778 | G | GGCGC | INDEL | MODIFIER | ECOLIN_23165 |
| 4889725 | TG | T | INDEL | MODIFIER | ECOLIN_23560 |
| 4902377 | TC | T | INDEL | HIGH | ECOLIN_23655 |
| 4976107 | TC | T | INDEL | HIGH | ECOLIN_23965 |
| 5000528 | G | GT | INDEL | MODIFIER | ECOLIN_24070 |
| 5010102 | TC | T | INDEL | HIGH | ECOLIN_24140 |
| 5041738 | CA | C | INDEL | HIGH | ECOLIN_24275 |
| 5068216 | TC | T | INDEL | MODIFIER | ECOLIN_24410 |
| 5077148 | TC | T | INDEL | MODIFIER | ECOLIN_24460 |
| 5080926 | T | C | SNP | MODIFIER | ECOLIN_24475 |
| 5113156 | CA | C | INDEL | MODIFIER | ECOLIN_24655 |
| 5125848 | T | C | SNP | MODIFIER | ECOLIN_24715 |
| 5153839 | A | G | SNP | MODIFIER | ECOLIN_24845 |
| 5153866 | G | A | SNP | MODIFIER | ECOLIN_24845 |
| 5153890 | A | T | SNP | MODIFIER | ECOLIN_24845 |
| 5163940 | CG | C | INDEL | MODIFIER | ECOLIN_24895 |
| 5191207 | G | T | SNP | MODIFIER | ECOLIN_25060 |
| 5249790 | T | C | SNP | MODERATE | ECOLIN_25390 |
| 5249835 | T | C | SNP | MODERATE | ECOLIN_25390 |
| 5249841 | G | T | SNP | MODERATE | ECOLIN_25390 |
| 5249844 | G | A | SNP | MODERATE | ECOLIN_25390 |
| 5249853 | A | G | SNP | MODERATE | ECOLIN_25390 |
| 5249856 | C | G | SNP | MODERATE | ECOLIN_25390 |
| 5249862 | C | G | SNP | MODERATE | ECOLIN_25390 |
| 5249877 | A | G | SNP | MODERATE | ECOLIN_25390 |
| 5249881 | C | T | SNP | LOW | ECOLIN_25390 |
| 5249886 | T | A | SNP | MODERATE | ECOLIN_25390 |
| 5249889 | C | G | SNP | MODERATE | ECOLIN_25390 |
| 5249892 | T | C | SNP | MODERATE | ECOLIN_25390 |
| 5249896 | G | A | SNP | LOW | ECOLIN_25390 |
| 5249910 | C | G | SNP | MODERATE | ECOLIN_25390 |
| 5249916 | C | T | SNP | MODERATE | ECOLIN_25390 |
| 5249931 | C | G | SNP | MODERATE | ECOLIN_25390 |
| 5249941 | T | C | SNP | LOW | ECOLIN_25390 |
| 5249943 | A | C | SNP | MODERATE | ECOLIN_25390 |
| 5249946 | C | A | SNP | MODERATE | ECOLIN_25390 |
| 5249949 | C | T | SNP | MODERATE | ECOLIN_25390 |
| 5249955 | G | A | SNP | MODERATE | ECOLIN_25390 |
| 5249958 | T | C | SNP | MODERATE | ECOLIN_25390 |
| 5249961 | C | G | SNP | MODERATE | ECOLIN_25390 |
| 5249970 | C | T | SNP | MODERATE | ECOLIN_25390 |
| 5249977 | C | T | SNP | LOW | ECOLIN_25390 |
| 5249981 | C | CG | INDEL | HIGH | ECOLIN_25390 |
| 5249984 | TA | T | INDEL | HIGH | ECOLIN_25390 |
| 5249988 | G | C | SNP | MODERATE | ECOLIN_25390 |
| 5249997 | G | A | SNP | MODERATE | ECOLIN_25390 |
| 5250003 | T | C | SNP | MODERATE | ECOLIN_25390 |
| 5250006 | G | C | SNP | MODERATE | ECOLIN_25390 |
| 5250009 | G | C | SNP | MODERATE | ECOLIN_25390 |
| 5250018 | A | G | SNP | MODERATE | ECOLIN_25390 |
| 5250021 | T | G | SNP | MODERATE | ECOLIN_25390 |
| 5250027 | T | C | SNP | MODERATE | ECOLIN_25390 |
| 5250034 | C | T | SNP | LOW | ECOLIN_25390 |
| 5250042 | G | A | SNP | MODERATE | ECOLIN_25390 |
| 5250402 | C | T | SNP | MODERATE | ECOLIN_25390 |
| 5250405 | C | T | SNP | MODERATE | ECOLIN_25390 |
| 5250417 | T | C | SNP | MODERATE | ECOLIN_25390 |
| 5250420 | C | G | SNP | MODERATE | ECOLIN_25390 |
| 5250424 | C | T | SNP | LOW | ECOLIN_25390 |
| 5250426 | A | G | SNP | MODERATE | ECOLIN_25390 |
| 5250432 | T | A | SNP | MODERATE | ECOLIN_25390 |
| 5250434 | G | A | SNP | MODERATE | ECOLIN_25390 |
| 5250435 | C | G | SNP | MODERATE | ECOLIN_25390 |
| 5250438 | A | G | SNP | MODERATE | ECOLIN_25390 |
| 5250441 | T | C | SNP | MODERATE | ECOLIN_25390 |
| 5250443 | A | G | SNP | MODERATE | ECOLIN_25390 |
| 5250468 | T | C | SNP | MODERATE | ECOLIN_25390 |
| 5250477 | A | G | SNP | MODERATE | ECOLIN_25390 |
| 5250478 | A | C | SNP | MODERATE | ECOLIN_25390 |
| 5250480 | A | G | SNP | MODERATE | ECOLIN_25390 |
| 5250483 | G | C | SNP | MODERATE | ECOLIN_25390 |
| 5250492 | C | T | SNP | MODERATE | ECOLIN_25390 |
| 5250504 | G | T | SNP | MODERATE | ECOLIN_25390 |
| 5250505 | C | A | SNP | LOW | ECOLIN_25390 |
| 5250507 | G | C | SNP | MODERATE | ECOLIN_25390 |
| 5250522 | T | TA | INDEL | HIGH | ECOLIN_25390 |
| 5250524 | GC | G | INDEL | HIGH | ECOLIN_25390 |
| 5250528 | A | G | SNP | HIGH | ECOLIN_25390 |
| 5250532 | A | G | SNP | MODERATE | ECOLIN_25390 |
| 5250534 | T | C | SNP | MODERATE | ECOLIN_25390 |
| 5250540 | C | G | SNP | MODERATE | ECOLIN_25390 |
| 5250549 | C | T | SNP | MODERATE | ECOLIN_25390 |
| 5250552 | T | C | SNP | MODERATE | ECOLIN_25390 |
| 5250561 | A | G | SNP | MODERATE | ECOLIN_25390 |
| 5250564 | G | A | SNP | MODERATE | ECOLIN_25390 |
| 5250573 | A | G | SNP | MODERATE | ECOLIN_25390 |
| 5250576 | T | G | SNP | MODERATE | ECOLIN_25390 |
| 5304113 | G | A | SNP | MODERATE | ECOLIN_25740 |
| 5304115 | A | G | SNP | MODERATE | ECOLIN_25740 |
| 5304118 | G | A | SNP | MODERATE | ECOLIN_25740 |
| 5304124 | T | C | SNP | MODERATE | ECOLIN_25740 |
| 5304250 | A | C | SNP | MODERATE | ECOLIN_25740 |
| 5332501 | CA | C | INDEL | MODIFIER | ECOLIN_25870 |
| 5345202 | T | C | SNP | MODIFIER | ECOLIN_25925 |
| 5345207 | A | G | SNP | MODIFIER | ECOLIN_25925 |
| 5345240 | C | G | SNP | MODIFIER | ECOLIN_25925 |
| 5345241 | TC | T | INDEL | MODIFIER | ECOLIN_25925 |
| 5345243 | A | T | SNP | MODIFIER | ECOLIN_25925 |
| 5345245 | G | GA | INDEL | MODIFIER | ECOLIN_25925 |
| 5345258 | A | AC | INDEL | MODIFIER | ECOLIN_25925 |
| 5345263 | CT | C | INDEL | MODIFIER | ECOLIN_25925 |
| 5345273 | A | G | SNP | MODIFIER | ECOLIN_25925 |
| 5345280 | CCG | C | INDEL | MODIFIER | ECOLIN_25925 |
| 5345284 | G | C | SNP | MODIFIER | ECOLIN_25925 |
| 5345285 | G | GAC | INDEL | MODIFIER | ECOLIN_25925 |
| 5345294 | A | G | SNP | MODIFIER | ECOLIN_25925 |
| 5346922 | TG | T | INDEL | MODIFIER | ECOLIN_25930 |
| 5374165 | G | T | SNP | LOW | ECOLIN_26080 |
| 5374166 | G | A | SNP | MODERATE | ECOLIN_26080 |
| 5374167 | C | A | SNP | MODERATE | ECOLIN_26080 |
| 5374172 | A | AG | INDEL | HIGH | ECOLIN_26080 |
| 5374175 | TC | T | INDEL | HIGH | ECOLIN_26080 |
| 5374184 | G | A | SNP | MODERATE | ECOLIN_26080 |
| 5374190 | A | C | SNP | MODERATE | ECOLIN_26080 |
| 5374195 | C | G | SNP | MODERATE | ECOLIN_26080 |
| 5374199 | A | T | SNP | MODERATE | ECOLIN_26080 |
| 5374202 | G | A | SNP | MODERATE | ECOLIN_26080 |
| 5374203 | T | A | SNP | MODERATE | ECOLIN_26080 |
| 5376958 | TG | T | INDEL | MODIFIER | ECOLIN_26075 |
| 5428111 | AC | A | INDEL | MODIFIER | ECOLIN_26325 |
| 5430346 | G | GTGCGTTTGCAGCAC | INDEL | MODIFIER | ECOLIN_26350 |
| | | (SEQ ID NO: 9) | | | |

### Discussion of Examples 1-3

An improved strain of E. coli Nissle was successfully engineered through adaptive laboratory evolution (ALE). The extensive characterization, including functional tests and genomic analysis, demonstrated the enhanced robustness of the engineered strain compared to the wildtype counterpart. These findings represent a significant step forward in harnessing the potential of E. coli Nissle for various applications, including biotechnology and therapeutic use.

These results underscore the efficacy of ALE as a powerful tool for enhancing microbial strains. By subjecting E. coli Nissle to a controlled evolutionary process, a strain that exhibits improved functionality under specific conditions was achieved. This success opens new avenues for tailoring bacterial strains to meet specific performance criteria.

The genomic analysis provided valuable insights into the genetic changes that underlie the improved performance of the engineered strain. This information can serve as a foundation for future research into the genetic determinants of robustness in E. coli Nissle and other microbial systems. Future research can also explore specific mutations and find what effect they have on the robustness of the cell.

### Example 4: CRISPR-Assisted Recombineering

CRISPR-aided recombineering works on the premise that since recombineering (generating mutations) is overall not a very efficient process, it can be aided by killing cells (via targeted double-stranded break) that have not obtained the mutation. Overall recombineering efficiency using CRISPR-Cas9, i.e., the ratio of transformed colonies that possess the mutation of interest, is a function of several factors, including transformation efficiency of the plasmid and insert, annealing of the homology arms, resistance to nucleases in the cell, induction of the λ-Red genes, and CRISPR killing efficiency. These experiments are designed to see if the overall recombineering efficiency is increased in the Zapped strain and to understand the possible mechanisms. The relevant parts used in these experiments are described in TABLE 5.

TABLE 5

| | |
|---|---|
| pCas9 | The plasmid encoding Cas9 (constitutive) and the λ-Red recombineering |
| | proteins (arabinose-inducible). This is pre-transformed into WT and |
| | Zapped before the start of the experiments. |
| pTargetF-mgsA | The plasmid encoding a guide RNA with an N20 corresponding to the |
| | mgsA gene in EcN. A “dummy” pTargetF plasmid contains an N20 not |
| | present in the EcN genome and therefore should not contribute to |
| | CRISPR-aided killing. |
| Insert | A double-stranded DNA cassette including a constitutive promoter, the |
| | GFP gene, and 500 bp homology arms on the 5′ and 3′ ends that |
| | correspond to the regions flanking the mgsA gene. |

During transformation, the insert, pTargetF, or both are electroporated and cells are plated on relevant antibiotics followed by colony counting. Cells that are fluorescent have the GFP cassette integrated into the genome, presumably at the mgsA site.

Overall Recombineering Efficiency—pTargetF mgsA with GFP Insert

The number of fluorescent colonies is compared in the wildtype and Zapped strains. Fluorescent colonies indicate successful homologous recombination facilitated by the fluorescent protein gene insert. The Zapped strain is expected to produce a significantly higher number of fluorescent colonies than the wildtype. This indicates greater recombineering efficiency, likely due to enhanced transformation efficiency, improved survival on electroporation, or a more efficient DNA repair system.

CRISPR Killing Efficiency—pTargetF mgsA without Insert

Without an insert, there should be no fluorescent colonies. Since bacteria typically lack non-homologous end joining (NHEJ) mechanisms, any transformants represent “escapers”-cells that survive CRISPR-based genome cutting. It is possible that the Zapped strain may exhibit a higher number of escapers because of its resilience to electroporation. However, since this condition measures survival post-CRISPR cutting without template-directed repair, the number of colonies may differ only slightly, and statistically insignificantly between wildtype and Zapped.

A Red Recombineering Efficiency—pTargetF Dummy with Insert

The outcomes for a dummy transformation with the fluorescent protein insert are compared in the wildtype and Zapped strains. Since the dummy pTargetF lacks a genomic target, no double-strand breaks should occur, and no CRISPR killing would occur. Fluorescent colonies would appear proportional to the rate of recombination. Both wildtype and Zapped are expected to have a small number (on the order of 1 in 10,000) fluorescent cells. However, the increased transformation efficiency of Zapped may allow more of the insert to enter the cell, which may increase the number of successful transformants.

Successful Plasmid Take-Up—pTargetF Dummy without Insert

This condition measures the baseline transformation efficiency by counting all colonies after transformation. Since there is no genomic target or fluorescent insert, all surviving colonies reflect successful plasmid uptake. The Zapped strain is expected to show a higher number of total transformants than the wildtype due to its higher transformation efficiency and robustness under identical experimental conditions.

### Calculation of Recombineering Efficiency

Recombineering efficiency will be calculated as:

$$Recombineering Efficiency = \frac{Number of recombinants ( fluorescent colonies from Target + Insert )}{Total cells plated ( colonies from pTargetF dummy without insert )}$$

### Escapers Comparison

The experiments will also help quantify “escapers”—colonies that survive CRISPR cutting without undergoing HDR. Escapers will be identified in conditions involving pTargetF-mgsA without an insert and dummy plasmids with inserts.

### Overall Expectations

Higher recombineering efficiency in Zapped: Zapped is expected to show a higher ratio of recombinants to total plated cells compared to wildtype due to increased transformation and recombineering efficiency.

Comparable escaper rates: Both strains are expected to have similar numbers of escapers, with the possibility of more survivors in the Zapped, but a higher overall efficiency. Non-homologous repair systems are expected to be similar in both strains.

### REFERENCES

Abil, Z., Ellefson, J. W., Gollihar, J. D., Watkins, E. & Ellington, A. D. Compartmentalized partnered replication for the directed evolution of genetic parts and circuits. Nat. Protoc. 12, 2493-2512 (2017).
Adediran, J. et al. An Escherichia coli Nissle 1917 Missense Mutant Colonizes the Streptomycin-Treated Mouse Intestine Better than the Wild Type but Is Not a Better Probiotic. Infect. Immun. 82, 670-682 (2014).
Ahmad, A. F., Dwivedi, G., O'Gara, F., Caparros-Martin, J., & Ward, N. C. The gut microbiome and cardiovascular disease: current knowledge and clinical potential. Am J Physiol Heart Circ Physiol. 317, H923-H938 (2019).
Bennett, A. F., Dao, K. M. & Lenski, R. E. Rapid evolution in response to high-temperature selection. Nature 346, 79-81 (1990).
Bordi, C., Théraulaz, L., Méjean, V. & Jourlin-Castelli, C. Anticipating an alkaline stress through the Tor phosphorelay system in Escherichia coli. Mol. Microbiol. 48, 211-223 (2003).
Bull, M. J. & Plummer, N. T. Part 1: The Human Gut Microbiome in Health and Disease. Integr. Med. Clin. J. 13, 17-22 (2014).
Dages, S., Zhi, X. & Leng, F. Fis protein forms DNA topological barriers to confine transcription-coupled DNA supercoiling in Escherichia coli. FEBS Lett. 594, 791-798 (2020).
Egan, S. M. & Schleif, R. F. A regulatory cascade in the induction of rhaBAD. J. Mol. Biol. 234, 87-98 (1993).
Elena, S. F. & Lenski, R. E. Evolution experiments with microorganisms: the dynamics and genetic bases of adaptation. Nat. Rev. Genet. 4, 457-469 (2003).
Evans, J., Morris, L. & Marchesi, J. The gut microbiome: The role of a virtual organ in the endocrinology of the host. J. Endocrinol. 218, (2013).
Galland, L. The Gut Microbiome and the Brain. J. Med. Food 17, 1261-1272 (2014).
Gibson, G. R. & Roberfroid, M. B. Dietary Modulation of the Human Colonic Microbiota: Introducing the Concept of Prebiotics. J. Nutr. 125, 1401-1412 (1995).
Gill, A. O. & Holley, R. A. Inhibition of membrane bound ATPases of Escherichia coli and Listeria monocytogenes by plant oil aromatics. Int. J. Food Microbiol. 111, 170-174 (2006).
Hancock, V., Dahl, M., & Klemm, P. Probiotic Escherichia coli strain Nissle 1917 outcompetes intestinal pathogens during biofilm formation. J Med Microbiol. 59, 392-399 (2010).
Kan, A., Gelfat, I., Emani, S., Praveschotinunt, P. & Joshi, N. S. Plasmid Vectors for in Vivo Selection-Free Use with the Probiotic E. coli Nissle 1917. ACS Synth. Biol. 10, 94-106 (2021).
Kim, K. et al. Adaptive laboratory evolution of Escherichia coli W enhances gamma-aminobutyric acid production using glycerol as the carbon source. Metab. Eng. 69, 59-72 (2022).
Lee, D.-H. & Palsson, B. Ø. Adaptive evolution of Escherichia coli K-12 MG1655 during growth on a Nonnative carbon source, L-1,2-propanediol. Appl. Environ. Microbiol. 76, 4158-4168 (2010).
Long, C. P., Gonzalez, J. E., Feist, A. M., Palsson, B. O. & Antoniewicz, M. R. Fast growth phenotype of E. coli K-12 from adaptive laboratory evolution does not require intracellular flux rewiring. Metab. Eng. 44, 100-107 (2017).
Lv, M. et al. Fis is a global regulator critical for modulation of virulence factor production and pathogenicity of Dickeya zeae. Sci. Rep. 8, 341 (2018).
Matson, M. M. et al. Adaptive laboratory evolution for improved tolerance of isobutyl acetate in Escherichia coli. Metab. Eng. 69, 50-58 (2022).
Nie, P., Li, Z., Wang, Y., Zhang, Y., Zhao, M., Luo, J., Du, S., Deng, Z., Chen, J., Wang, Y., Chen, S., & Wang, L. Gut microbiome interventions in human health and diseases. Med Res Rev. 39, 2286-231 (2019).
O'Toole, P. W., Marchesi, J. R. & Hill, C. Next-generation probiotics: the spectrum from probiotics to live biotherapeutics. Nat. Microbiol. 2, 17057 (2017).
Ou, B. et al. Genetic engineering of probiotic Escherichia coli Nissle 1917 for clinical application. Appl. Microbiol. Biotechnol. 100, 8693-8699 (2016).
Pandey, Kavita. R., Naik, Suresh. R. & Vakil, Babu. V. Probiotics, prebiotics and synbiotics—a review. J. Food Sci. Technol. 52, 7577-7587 (2015).
Raeside, C. et al. Large Chromosomal Rearrangements during a Long-Term Evolution Experiment with Escherichia coli. mBio 5, 10.1128/mbio.01377-14 (2014).
Rosenberg, S. M., Thulin, C. & Harris, R. S. Transient and Heritable Mutators in Adaptive Evolution in the Lab and in Nature. Genetics 148, 1559-1566 (1998).
Rothstein S. M., Sen S, Mansell T. J. Towards high-throughput genome engineering in lactic acid bacteria. Curr Opin Biotechnol. 61, 181-188 (2020).
Sandberg, T. E., Salazar, M. J., Weng, L. L., Palsson, B. O. & Feist, A. M. The emergence of adaptive laboratory evolution as an efficient tool for biological discovery and industrial biotechnology. Metab. Eng. 56, 1-16 (2019).
Schleif, R. AraC protein: A love-hate relationship. BioEssays 25, 274-282 (2003).
Schneider, R. et al. An architectural role of the Escherichia coli chromatin protein FIS in organising DNA. Nucleic Acids Res. 29, 5107-5114 (2001).
Sekirov, I., Russell, S. L., Antunes, L. C. M. & Finlay, B. B. Gut Microbiota in Health and Disease. Physiol. Rev. 90, 859-904 (2010).
Simon, G., Méjean, V., Jourlin, C., Chippaux, M. & Pascal, M. C. The torR gene of Escherichia coli encodes a response regulator protein involved in the expression of the trimethylamine N-oxide reductase genes. J. Bacteriol. 176, 5601-5606 (1994).
Sonnenborn, U. 100 years of E. coli strain Nissle 1917. OUPblog blog.oup.com/2017/04/e-coli-strain-nissle-1917-timeline/(2017).
Ting, W.-W. & Ng, I.-S. Adaptive laboratory evolution and metabolic regulation of genetic Escherichia coli W3110 toward low-carbon footprint production of 5-aminolevulinic acid. J. Taiwan Inst. Chem. Eng. 141, 104612 (2022).
Tobin, J. F. & Schleif, R. F. Positive regulation of the Escherichia coli L-rhamnose operon is mediated by the products of tandemly repeated regulatory genes. J. Mol. Biol. 196, 789-799 (1987).
Tobin, J. F. & Schleif, R. F. Purification and properties of RhaR, the positive regulator of the 1-rhamnose operons of Escherichia coli. J. Mol. Biol. 211, 75-89 (1990).
Wang, H. H. et al. Programming cells by multiplex genome engineering and accelerated evolution. Nature 460, 894-898 (2009).
Weinstein-Fischer, D. & Altuvia, S. Differential regulation of Escherichia coli topoisomerase I by Fis. Mol. Microbiol. 63, 1131-1144 (2007).
Wielgoss, S. et al. Mutation Rate Inferred From Synonymous Substitutions in a Long-Term Evolution Experiment With Escherichia coli. G3 GenesGenomesGenetics 1, 183-186 (2011).
Xu, J., Zhou, L., Yin, M. & Zhou, Z. Novel Mode Engineering for β-Alanine Production in Escherichia coli with the Guide of Adaptive Laboratory Evolution. Microorganisms 9, 600 (2021).
Yeowell, H. N. & White, J. R. Iron requirement in the bactericidal mechanism of streptonigrin. Antimicrob. Agents Chemother. 22, 961-968 (1982).
Zainuddin, H. S., Bai, Y. & Mansell, T. J. CRISPR-based curing and analysis of metabolic burden of cryptic plasmids in Escherichia coli Nissle 1917. Eng. Life Sci. 19, 478-485 (2019).

What is claimed is:

1. An engineered E. coli Nissle strain having increased transformation efficiency relative to wild type E. coli Nissle, deposited under ATCC accession number PTA-______, or a derivative thereof.

2. The engineered E. coli Nissle strain of claim 1, wherein the derivative comprises a genome sequence having at least 95%, at least 98%, at least 99%, at least 99.5% at least 99.6% at least 99.7% at least 99.8%, or at least 99.9% sequence identity to SEQ ID NO: 2.

3. The engineered E. coli Nissle strain of claim 1, wherein the derivative comprises all of the mutations set forth in Table 5.

4. The engineered E. coli Nissle strain of claim 1, wherein the derivative further comprises a heterologous polynucleotide.

5. The engineered E. coli Nissle strain of claim 1, wherein the derivative further comprises one or more additional mutations.

6. A method of obtaining an E. coli transformant comprising transforming a heterologous polynucleotide into the engineered E. coli Nissle strain deposited under ATCC accession number PTA-______.

7. The method of claim 6, wherein the transforming comprises electroporation or heat shock.

8. The method of claim 6, wherein the transformation efficiency is increased at least 2-fold, at least 3-fold, at least 4-fold, at least 5-fold, at least 6-fold, at least 7-fold, at least 8-fold, at least 9-fold, or at least 10-fold relative to the wild type E. coli Nissle strain.

9. A probiotic composition comprising the engineered E. coli Nissle strain deposited under ATCC accession number PTA-______, or a derivative thereof.

10. The probiotic composition of claim 9, further comprising a prebiotic.

11. The probiotic composition of claim 9, further comprising pharmaceutically acceptable carrier.

12. The probiotic composition of claim 9, wherein the composition is formulated as a powder, a granule, a tablet, a capsule, a liquid suspension, a paste, or a syrup.

13. The probiotic composition of claim 9, wherein the derivative comprises a genome sequence having at least 95%, at least 98%, at least 99%, at least 99.5% at least 99.6% at least 99.7% at least 99.8%, or at least 99.9% sequence identity to SEQ ID NO: 2.

14. The probiotic composition of claim 9, wherein the derivative comprises all of the mutations set forth in Table 5.

15. The probiotic composition of claim 9, wherein the derivative further comprises a heterologous polynucleotide.

16. The probiotic composition of claim 9, wherein the derivative further comprises one or more additional mutations.

17. A method for establishing or maintaining a healthy gastrointestinal microbiota or reducing the effects of a gastrointestinal disorder in a subject, the method comprising: administering to the subject a composition comprising the engineered E. coli Nissle strain deposited under ATCC accession number PTA-______, or a derivative thereof.

18. The method of claim 17, wherein the composition further comprises a prebiotic.

19. The method of claim 17, wherein the composition further comprises pharmaceutically acceptable carrier.

20. The method of claim 17, wherein the composition is formulated as a powder, a granule, a tablet, a capsule, a liquid suspension, a paste, or a syrup.

21. The method of claim 17, wherein the derivative comprises a genome sequence having at least 95%, at least 98%, at least 99%, at least 99.5% at least 99.6% at least 99.7% at least 99.8%, or at least 99.9% sequence identity to SEQ ID NO: 2.

22. The method of claim 17, wherein the derivative comprises all of the mutations set forth in Table 5.

23. The method of claim 17, wherein the derivative further comprises a heterologous polynucleotide.

24. The method of claim 17, wherein the derivative further comprises one or more additional mutations.

25. The method of claim 17, wherein the composition is administered orally or rectally.

26. The method of claim 17, wherein the subject is a mammal.

27. A method of altering a target nucleic acid sequence within an E. coli cell, the method comprising:
providing a cell of the engineered E. coli Nissle strain of claim 1 with a Cas protein, a guide RNA, a donor nucleic acid sequence, and a recombinase.

28. The method of claim 27, wherein the guide RNA and the Cas protein co-localize at the target nucleic acid sequence, the Cas protein cleaves the target nucleic acid sequence, and the donor nucleic acid sequence is inserted into the target nucleic acid sequence.

29. The method of claim 27, wherein the recombinase is a λ-Red recombination system.
